(19) United States
(12) Patent Application Publication
(10) Pub. No.: US 2019/0083642 A1
(43) Pub. Date: Mar. 21, 2019

(54) TREATMENTS UTILIZING A POLYMER-PROTEIN CONJUGATE

(71) Applicant: Regentis Biomaterials Ltd., Or-Akiva (IL)

(72) Inventors: Yonatan SHACHAF, Haifa (IL); Aharon WECHSLER, Shoham (IL)

(73) Assignee: Regentis Biomaterials Ltd., Or-Akiva (IL)

(21) Appl. No.: 16/088,454

(22) PCT Filed: Mar. 30, 2017
(86) PCT No.: PCT/IL2017/050397

§ 371 Date: Sep. 26, 2018

Related U.S. Application Data
(60) Provisional application No. 62/315,043, filed on Mar. 30, 2016.

(51) Int. Cl. A61K 47/64; A61K 47/60; A61K 47/69; A61K 31/728; A61K 9/00; A61K 9/08; A61L 27/52; A61P 19/02; A61P 19/04

(52) U.S. Cl. CPC A61K 47/6435; A61K 47/60; A61K 47/6903; A61K 31/728; A61K 9/0019; A61L 2430/06; A61L 27/52; A61P 19/02; A61P 19/04; A61L 2430/02; A61K 9/08

(57) ABSTRACT

A composition for use in treating a condition associated with degeneration of articular cartilage and/or with subchondral bone loss is disclosed herein. The composition comprises a conjugate which comprises a polypeptide having attached thereto at least two polymeric moieties, at least one of the polymeric moieties exhibiting a reverse thermal gelation. Further disclosed is a composition comprising the aforementioned conjugate along with a hyaluronic acid, an anti-inflammatory agent, an analgesic, a growth factor, a blood fraction, a nucleic acid, and/or a cell, the composition being an aqueous composition which forms a hydrogel at a temperature in a range of from 32° C. to 37° C., as well as a method utilizing such a composition comprising a nucleic acid for effecting gene delivery.

### FIELD AND BACKGROUND OF THE INVENTION

The present invention, in some embodiments thereof, relates to therapy, and more particularly, but not exclusively, to compositions comprising a polymer-protein conjugate and uses thereof in therapeutic applications such as, for example, in the treatment of degeneration of articular cartilage and/or subchondral bone loss, and conditions associated therewith, such as arthritis.

Cartilage and subchondral bone (i.e., bone beneath cartilage) are dynamic stress bearing structures that play complementary roles in load-bearing of joints. Subchondral bone supports overlying articular cartilage and distributes mechanical loads across joint surfaces [Li et al., Arthritis Res Ther 2013, 15:223].

Osteoarthritis (OA) is the most common joint disease with prevalence of over 20 million in the United States alone, causing disability and reduction of quality of life and participation in social activity. It involves cartilage loss, subchondral bone changes, synovial inflammation and meniscus degeneration [Favero et al., RMD Open 2015, 1(Suppl 1):e000062; Loeser et al., Arthritis Rheum 2012, 64:1697-1707]. Risk factors for osteoarthritis include age, gender, obesity, occupation, trauma, atheromatous vascular disease and immobilization [Alexander, Skeletal Radiol 2004, 33:321-324]. OA can originate from inflammation, metabolic and mechanical causes. OA may arise as a result of articular cartilage breakdown; or conversely, subchondral bone sclerosis may actually precede cartilage degeneration and loss [Moskowitz et al., Am J Orthop (Belle Mead N.J.) 2004, 33(Suppl 2):5-9; Imhof et al., Invest. Radiol 2000, 35:581-588]. It is associated with progressive damage to the articular cartilage with involvement of the subchondral bone, osteophyte formation, thickening of the joint capsule and synovitis, causing discomfort and pain in the affected joint. In many cases knee replacement will be necessary as a final method of restoring function and decreasing pain [Cuervo et al., International Journal of Orthopaedics 2015, 210-218; Radin, J Rheumatol 2005, 32:1136-1138].

In early stages of OA in humans, elevated bone remodeling and subchondral bone loss is observed, and is considered as a factor of OA progression [Bettica et al., Arthritis Rheum 2002, 46:3178-3184]. The cavitary lesions in the subchondral bone, referred to as “subchondral bone cysts”, are commonly reported in patients with OA, and recent evidence suggests that patients with subchondral bone cysts (SBC) have greater disease severity and pain, and a higher risk of joint replacement [Tanamas et al., Arthritis Res Ther 2010, 12:R58].

Current management of OA includes reducing overloading of joints by weight control and exercise, systemic or topical non-steroid anti-inflammatory drugs (NSAIDS), analgesia (e.g., paracetamol), topical capsaicin, oral and topical opioids, noradrenaline and serotonin reuptake inhibitors (e.g., duloxetine), complementary glucosamine and chondroitin sulfate [Yu & Hunter, Aust Prescr 2015, 38:115-119].

OA is also treated by intra-articular injections of therapeutics such as corticosteroids, hyaluronic acid (HA)-based viscosupplements and platelet-rich plasma (PRP) [Yu & Hunter, Aust Prescr 2015, 38:115-119; Evans et al., Nat Rev Rheumatol 2014, 10:11-22]. This mode of delivery suffers from rapid egress of injected materials from joint space to the circulation or via the lymphatic system, depending on size of the injected molecule [Evans et al., Nat Rev Rheumatol 2014, 10:11-22]. Corticosteroids are effective, but prolonged use is not advisable due to possible adverse effects and acceleration of the disease.

HA-based viscosupplements are commonly delivered via intra-articular injection, and may include cross-linked HA (e.g., Synvisc-One®) or non-cross-linked HA (e.g., Arthrease®). Their use is based on the observation that the concentration and molecular weight of HA in osteoarthritic joints is decreased, which is believed to lead to loss of lubrication and shock absorption [Ammar et al., Rev Bras Ortop 2015, 50:489-494; Strauss et al., Am J Sports Med 2009, 37:1636-1644]. Nevertheless, recent systemic reviews and meta-analysis of numerous clinical trials using HA viscosupplements indicate that their efficacy is questionable [Jevsevar et al., J Bone Joint Surg Am 2015, 97:2047-2060; Ammar et al., Rev Bras Ortop 2015, 50:489-494; Evans et al., Nat Rev Rheumatol 2014, 10:11-22]. A drawback of HA-based viscosupplements is that they follow the same fate as the endogenous HA which they intend to supplement, i.e., a relatively short half-life which ranges from several hours to few days [Wen, Am Fam Physician 2000, 62:565-70; Larsen et al., J Biomed Mater Res B Appl Biomater 2012, 100:457-462; Benke & Shaffer, Curr Pain Headache Rep 2009, 13:440-446].

Intra-articular injection of platelet-rich plasma (PRP) has been reported to result in significantly better outcome vs. HA in several clinical studies [Meheux et al., Arthroscopy 2016, 32:495-505; Xie et al., Arthritis Res Ther 2014, 16:204; Cuervo et al., International Journal of Orthopaedics 2015, 210-218].

Saito et al. [Clin Exp Rheumatol 2009, 27:201-207] describes a hydrogel containing PRP for sustainably releasing growth factors in the PRP.

Additional approaches include intra-articular injections of stem cells; antibodies and receptor antagonists to pro-inflammatory cytokines, such as anti-TNF and anti-IL1β antibodies and IL1-receptor antagonist; and growth factors such as bone morphogenetic protein 7 (BMP-7) and fibroblast growth factor 18 (FGF-18)) [Cuervo et al., International Journal of Orthopaedics 2015, 210-218].

Another approach under investigation involves intra-articular delivery of genes via viral or non-viral vectors, either directly or via administration of cells that were modified genetically ex vivo [Madry et al., Cartilage 2011, 2:201-225; Madry & Cucchiarini, J Gene Med 2013, 15:343-355; Evans et al., Transl Res 2013, 161:205-2016]. In Phase II clinical trials, improved outcomes have been reported following intra-articular injection of either an adeno-associated virus (AAV) vector encoding for etanercept in rheumatoid arthritis patients [Mease et al., J Rheumatol 2010, 37:692-703] or genetically engineered chondrocytes which produce TGF-β in osteoarthritis patients [Ha et al., Hum Gene Ther Clin Dev 2015, 26:125-130].

International Patent Application Publication WO 2011/073991 describes compositions comprising conjugates of a polymer such as F127 poloxamer with a protein such as fibrinogen, as well as reverse thermal gelation exhibited by such compositions, their compatibility with seeded cells, and their use for applications such as cell growth and tissue formation. Properties and uses of fibrinogen-F127 poloxamer conjugates are further described by Shachaf et al. [Biomaterials 2010, 31:2836-2847] and Frisman et al. [Langmuir 2011, 27:6977-6986].

Rothenfluh et al. [Nat Mater 2008, 7:248-254] describes conjugation of a cartilage-binding hexapeptide to an F127 poloxamer-based nanoparticle, and use of the conjugate to deliver a drug encapsulated within the nanoparticle to articular cartilage.

Additional background art includes Almany and Seliktar [Biomaterials 2005, 26:2467-2477], Eguiluz et al. [Biomacromolecules 2015, 16:2884-2894], Evans et al. [Nat Rev Rheumatol 2014, 10:11-22], Gobbi et al. [Knee Surg Sports Traumatol Arthrosc 2015, 23:2170-2177], Gonen-Wadmany et al. [Biomaterials 2007, 28:3876-3886], Jay & Waller [Matrix Biol 2014, 39:17-24], Peled et al. [Biomed Mater Res A 2007, 80:874-884], and Seliktar [Ann NY Acad Sci 2005, 1047:386-394]; International Patent Application Publications WO 2005/061018, WO 2008/126092 and WO 2014/207749; U.S. Patent Application Publication No. 2011/0125156; and U.S. Pat. Nos. 8,007,774 and 7,842,667.

### SUMMARY OF THE INVENTION

According to an aspect of some embodiments of the invention, there is provided a composition comprising a conjugate which comprises a polypeptide having attached thereto at least two polymeric moieties, at least one of the polymeric moieties exhibiting a reverse thermal gelation, the composition being for use in treating a condition associated with degeneration of articular cartilage and/or with subchondral bone loss.

According to an aspect of some embodiments of the invention, there is provided a pharmaceutical composition comprising:

a conjugate which comprises a polypeptide having attached thereto at least two polymeric moieties, at least one of the polymeric moieties exhibiting a reverse thermal gelation; and

at least one additional therapeutically active agent selected from the group consisting of a hyaluronic acid, an anti-inflammatory agent, an analgesic, a growth factor, a blood fraction, a nucleic acid, and a cell,

the composition being an aqueous composition which forms a hydrogel at a temperature in a range of from 32° C. to 37° C.

According to an aspect of some embodiments of the invention, there is provided a method of effecting gene delivery, the method comprising contacting at least one cell with a composition described herein, the composition comprising a nucleic acid described herein, and the nucleic acid comprising the abovementioned gene, thereby effecting delivery of the gene to at least one cell.

According to some embodiments of the invention, the method is effected ex vivo.

According to some embodiments of any of the embodiments of the invention, treating comprises intra-articular administration of the composition.

According to some embodiments of the invention, the administration comprises intra-articular injection.

According to some embodiments of the invention, the degeneration of articular cartilage and/or subchondral bone loss is associated with friction at a surface of the articular cartilage.

According to some embodiments of the invention, the condition is associated with a subchondral bone cyst.

According to some embodiments of the invention, treating comprises injecting the composition into said bone cyst. According to some embodiments of the invention, the composition is characterized by a static coefficient of friction which is less than 0.2.

According to some embodiments of the invention, the degeneration is associated with an inflammation.

According to some embodiments of the invention, the composition reduces degeneration of cartilage induced by inflammation.

According to some embodiments of the invention, the composition is characterized by water uptake of less than 20 weight percents upon incubation with an aqueous liquid for 48 hours at a temperature of 37° C.

According to some embodiments of the invention, the composition comprises an aqueous solution of the conjugate.

According to some embodiments of the invention, the composition forms a hydrogel at a temperature in a range of from 32° C. to 37° C.

According to some embodiments of the invention, a shear storage modulus of the hydrogel is at least 15 Pa.

According to some embodiments of the invention, the composition is capable of undergoing a reverse thermal gelation.

According to some embodiments of the invention, the composition further comprises at least one additional therapeutically active agent.

According to some embodiments of the invention, the additional therapeutically active agent is selected from the group consisting of a hyaluronic acid, an anti-inflammatory agent, an analgesic, a growth factor, a blood fraction, a nucleic acid, and a cell.

According to some embodiments of the invention, wherein at least one additional therapeutically active agent is selected from the group consisting of a hyaluronic acid, a blood fraction, and a nucleic acid.

According to some embodiments of the invention, at least 20 weight percents of the composition is the blood fraction.

According to some embodiments of the invention, the blood fraction is selected from the group consisting of platelet-rich plasma and platelet-poor plasma.

According to some embodiments of the invention, the composition is capable of sustained release of the therapeutically active agent.

According to some embodiments of the invention, the sustained release is characterized by retention of at least 20% of the therapeutically active agent upon incubation for 48 hours in an aqueous environment.

According to some embodiments of the invention, the condition is arthritis.

According to some embodiments of the invention, the arthritis is osteoarthritis.

According to some embodiments of the invention, at least a portion of the articular cartilage and/or the subchondral bone is in a synovial joint.

According to some embodiments of the invention, the composition is for use in treating a condition treatable by a therapeutically active agent comprised by the composition.

According to some embodiments of the invention, the condition is treatable by local administration of the therapeutically active agent, and the treating comprises local administration of the composition.

According to some embodiments of the invention, the at least one therapeutically active agent comprises a blood fraction described herein, and the condition is selected from the group consisting of arthritis, nerve injury, tendinitis, muscle injury, bone injury, and surgical injury.

According to some embodiments of the invention, the treating comprises delivery of a gene comprised by a nucleic acid described herein to cells, wherein the condition is treatable by expression of the gene in vivo.

According to some embodiments of the invention, the at least one therapeutically active agent comprises hyaluronic acid, and the condition is arthritis.

According to some embodiments of the invention, the condition is treatable by a substance produced by the cell.

According to some embodiments of any of the embodiments of the invention, the polypeptide is at least 20 amino acids in length.

According to some embodiments of the invention, the polypeptide is capable of adhering to cartilage.

According to some embodiments of the invention, the polypeptide exhibits greater affinity to damaged cartilage than to undamaged cartilage.

According to some embodiments of the invention, the polypeptide comprises a protein or a fragment thereof.

According to some embodiments of the invention, the polypeptide is selected from the group consisting of fibrinogen, collagen, fibronectin, elastin, fibrillin, fibulin, laminin, albumin, von Willebrand factor and gelatin, and fragments thereof.

According to some embodiments of the invention, the polypeptide comprises a fibrinogen or a fragment thereof.

According to some embodiments of the invention, the protein is denatured.

According to some embodiments of the invention, the polypeptide is a denatured fibrinogen.

According to some embodiments of the invention, each of the polymeric moieties exhibits a reverse thermal gelation.

According to some embodiments of the invention, the polymeric moieties comprise a synthetic polymer.

According to some embodiments of the invention, at least one of the polymeric moieties comprises a poloxamer (poly(ethylene oxide-propylene oxide) copolymer).

According to some embodiments of the invention, each of the polymeric moieties comprises a poloxamer.

According to some embodiments of the invention, the poloxamer is F127 poloxamer.

According to some embodiments of the invention, at least one of the polymeric moieties further comprises at least one cross-linking moiety capable of covalently cross-linking the conjugate with a protein in vivo.

According to some embodiments of the invention, the cross-linking moiety is selected from the group consisting of an acrylate, a methacrylate, an acrylamide, a methacrylamide, and a vinyl sulfone.

According to some embodiments of the invention, the polypeptide is denatured fibrinogen and the polymeric moieties comprise F127 poloxamer.

According to some embodiments of the invention, the conjugate comprises F127 poloxamer diacrylate moieties, wherein an acrylate group of each of the F127 poloxamer diacrylate moieties is attached to a cysteine residue of the fibrinogen.

According to some embodiments of any of the embodiments of the invention, the composition is an injectable composition.

According to some embodiments of any of the embodiments of the invention, at least one cell is encapsulated by the composition and/or cultured on a surface of the composition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents images showing the fluidity of an exemplary polymer-protein composition according to some embodiments of the invention (GelrinV) at 22° C. and its gelation at 37° C. (composition dyed for clarity).

FIGS. 2A and 2B present phase-contrast microscopy (FIG. 2A) and fluorescent microscopy (FIG. 2B) images showing bovine cartilage explants with circular abrasions (1.5 mm diameter), following incubation for 3 days with fluorescein isothiocyanate-labeled F127-fibrinogen.

FIG. 3 presents images of histological cross-sections of cartilage-like chondrocyte pellets treated with an exemplary polymer-protein composition according to some embodiments of the invention (GelrinV) in the presence of 1 ng/ml IL-1β (upper panels show collagen II staining and lower panels each show fibrinogen staining in the corresponding region).

FIG. 4 presents images of sections of chondrocyte pellets stained for collagen II following exposure to 0.5 ng/ml IL-1β alone or along with Synvisc-One® viscosupplement or an exemplary polymer-protein composition according to some embodiments of the invention (GelrinV) (control sample was not exposed to IL-1β).

FIG. 5 is a bar graph showing levels of glycosaminoglycans (as a percentage of untreated control) in chondrocyte pellets following treatment for 4 days with IL-1β with and without an exemplary polymer-protein composition according to some embodiments of the invention (GelrinV) (results represent mean±SEM values of at least 6 samples).

FIGS. 6A and 6B are each bar graphs showing water uptake of an exemplary polymer-protein gel composition according to some embodiments of the invention (GelrinV), a hyaluronic acid-based viscosupplement gel (Synvisc-One® in FIG. 6A, Arthrease® in FIG. 6B), and a 1:1 mixture of the viscosupplement and GelrinV, following incubation in PBS (at a 1:3.5 ratio of gel to PBS) for 48 hours at 37° C. (results represent mean±STDEV values for 3 samples).

FIG. 7 is a bar graph showing maximal shear storage modulus (G′) of Synvisc-One® viscosupplement (100% HA), an exemplary polymer-protein composition according to some embodiments of the invention (GelrinV) and a 1:1 mixture of Synvisc-One® viscosupplement and GelrinV (HA:GelrinV (1:1)) before (T=0) and after (T=48 hrs) incubation for 48 hours at 37° C. in PBS in the absence or presence of 300 μg/ml hyaluronidase (HAase) (results represent mean±STDEV values for 3 samples).

FIG. 8 is a bar graph showing static coefficients of friction for an exemplary composition according to some embodiments of the invention (GelrinV) and for Synvisc-One® viscosupplement (results shown are mean of 4 samples).

FIG. 9 is a graph showing kinetic coefficients of friction for an exemplary composition according to some embodiments of the invention (GelrinV) and for Synvisc-One® viscosupplement, as a function of sliding velocity (in a sliding velocity range of from 2 to 81 mm per second, results shown are mean of 4 samples).

FIG. 10 is a scheme depicting an articular cartilage surface (shaded blue) exhibiting erosion of cartilage and a mechanism by which a conjugate comprising poloxamer (Pluronic-F127) and fibrinogen moieties can adhere to the cartilage surface via the fibrinogen moiety and provide lubrication via the poloxamer moiety, according to optional embodiments of the invention.

FIG. 11 presents a timeline describing an experimental protocol using a surgically induced arthritis rat model, including evaluation of pain by von Frey method (VF) and gait analysis.

FIG. 12 presents images of representative histological cross sections showing rat joints stained with toluidine blue following treatment with an exemplary composition according to some embodiments of the invention (GelrinV), Synvisc-One® viscosupplement or phosphate buffer saline (PBS) (arrow indicates location of cartilage degeneration through more than 50% of the cartilage thickness).

FIG. 13 is a bar graph showing the width of substantial cartilage degeneration in rat joints following treatment with an exemplary composition according to some embodiments of the invention (GelrinV) or with Synvisc-One® viscosupplement, as a percentage of substantial cartilage degeneration width following treatment with phosphate buffer saline (PBS) (results represent mean±SE values of 10 samples).

FIG. 14 presents images of a representative histological cross section (at different magnifications) of a rat joint two intra-articular injections (14 and 28 days prior) of an exemplary composition according to some embodiments of the invention (GelrinV), showing the presence of GelrinV conjugate molecules indicated by anti-polyethylene glycol antibodies (red staining) (sample also stained blue/violet with hematoxylin; right panel represents area indicated by dashed rectangle in middle panel, and middle panel represents area indicated by dashed rectangle in left panel).

FIG. 15 is a bar graph showing mean allodynia (according to von Frey pain protocol) in paws of rats in an osteoarthritic model, following treatment with an exemplary composition according to some embodiments of the invention (GelrinV), Synvisc-One® viscosupplement or phosphate buffer saline (PBS).

FIGS. 16A and 16B are bar graphs showing the effects of treatment with an exemplary composition according to some embodiments of the invention (GelrinV), Synvisc-One® viscosupplement or phosphate buffer saline (PBS) on gaits of rats, evaluated as mean gait score (FIG. 16A; 0 score=normal gait, maximal score of 6=hopping) and as gait deficiency percentage (FIG. 16B).

FIG. 17 is a graph showing the shear storage modulus (G′) of homogenous solutions formed by mixing (at a temperature below 20° C.) an exemplary composition (GelrinV) at a 1:1 volume ratio with non-activated human platelet rich plasma (PRP), platelet poor plasma (PPP) or phosphate buffer saline (PBS).

FIG. 18 presents images of Cy3-labeled DNA plasmid entrapped in an exemplary composition (GelrinV) either as free (“naked”) plasmid or in complex with polyethylenimine (PEI) or PolyJet™ as a function of time, after mixing 300 μl of the composition with a solution (100 μl) of Cy3-labeled plasmid DNA (0.5 μg) and non-labeled plasmid DNA (0.5 μg) at 4° C., followed by incubation at 37° C. with addition of 100 μl PBS.

FIGS. 19A-19F present an image of a polymer-protein composition (GelrinV) comprising green fluorescent protein (GFP) plasmid nano-complexes in culture medium according to some embodiments of the invention (FIG. 19A) and fluorescent microscopy images of C2C12 myoblast cells; cells were encapsulated in GelrinV following pre-incubation with nano-complexes (FIG. 19B) or concomitantly with nano-complexes (FIG. 19C), or cells were seeded as a 2D layer over a layer of GelrinV with nano-complexes in a plastic culture plate (FIG. 19E) or tube (FIG. 19F) system, or GelrinV with nano-complexes was deposited above the cell layer (FIG. 19D).

FIG. 20 presents images of fluorescent microscopy images of C2C12 myoblast cells seeded as a 2D layer over a layer of an exemplary polymer-protein composition (GelrinV) comprising green fluorescent protein (GFP) plasmid nano-complexes; 3 different C2C12 cultures (arbitrarily numbered 1, 2 and 3) are shown under two different conditions: cultures with no wash (upper panels) and cultures following extensive wash (lower panels).

### DESCRIPTION OF SPECIFIC EMBODIMENTS OF THE INVENTION

The present invention, in some embodiments thereof, relates to therapy, and more particularly, but not exclusively, to compositions comprising a polymer-protein conjugate and uses thereof in therapeutic applications such as, for example, in the treatment of degeneration of articular cartilage and/or subchondral bone loss, and conditions associated therewith, such as arthritis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In a search for methodologies for generating more effective treatment of conditions such as arthritis, the present inventors have envisioned that compositions which comprise polymer-protein conjugates that exhibit reverse thermal gelation can be used to lubricate joints, and thereby protect cartilage against degeneration, and/or administer to subchondral bone cysts, while also being relatively easy to administer in a fluid (non-gel) form.

While reducing the present invention to practice, the inventors of the present invention have surprisingly uncovered that polymer-protein conjugates advantageously adhere to cartilage, protect cartilage against inflammatory effects, resist dilution in an aqueous environment, and exhibit superior lubricating and rheological properties in comparison with standard hyaluronic acid viscosupplements used for treating joints.

The inventors of the present inventors have conceived, and demonstrated, that these properties render compositions comprising such polymer-protein conjugates advantageous for a variety of applications, including lubrication of articular cartilage surfaces, as well as facilitating delivery of a therapeutically active agent, and gene delivery.

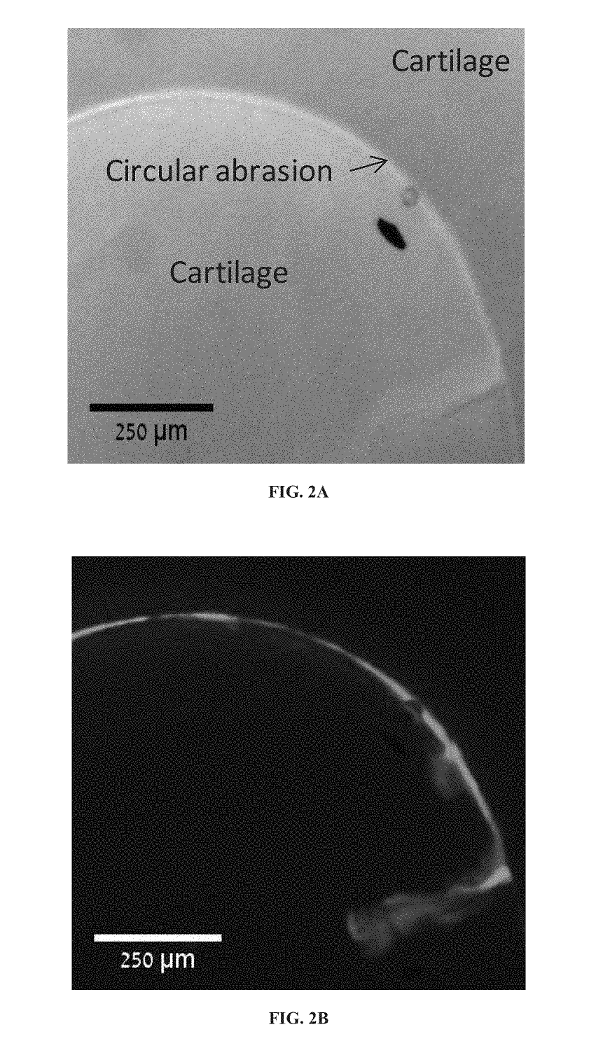

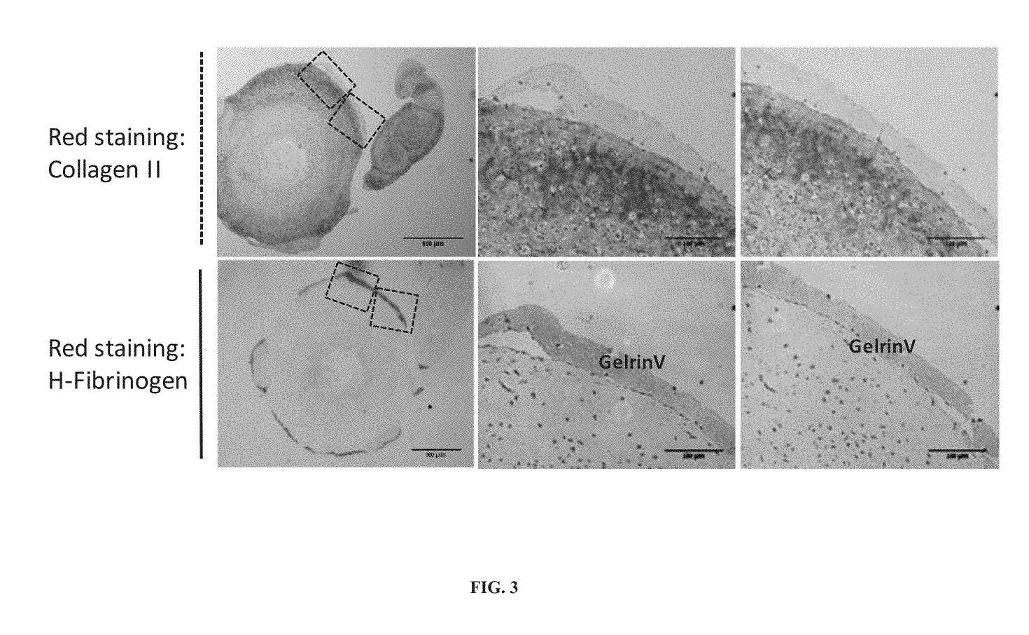

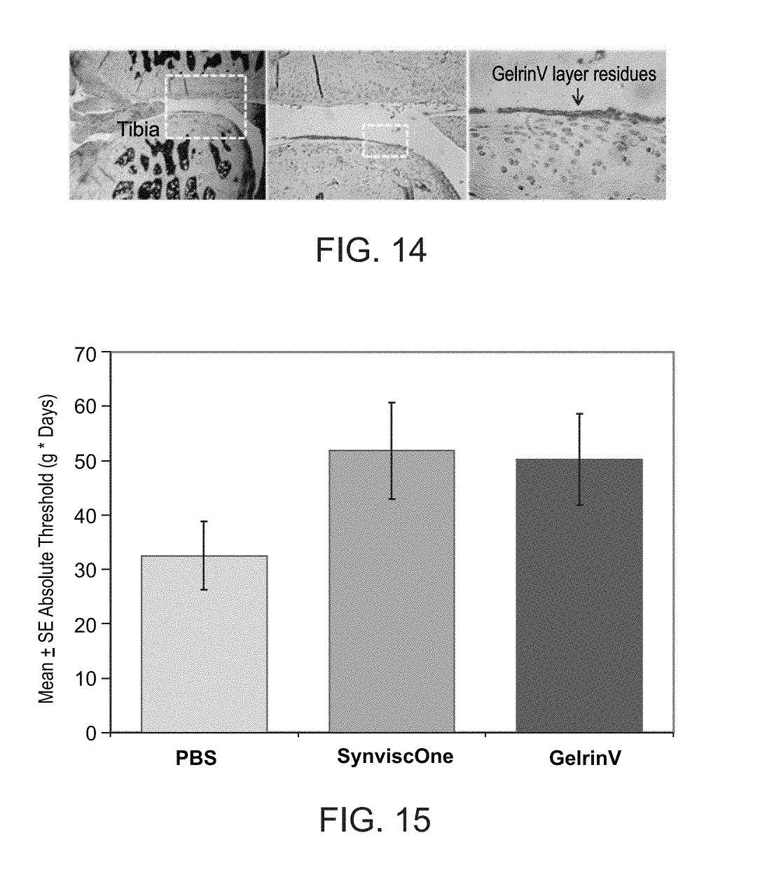

Referring now to the drawings, FIGS. 2A-3 show that exemplary poloxamer-fibrinogen conjugates adhere to cartilage in an in vitro model. FIGS. 2A and 2B further show that the conjugates selectively adhere to damaged cartilage. FIG. 14 shows that the conjugates adhere to cartilage in arthritic joints in vivo.

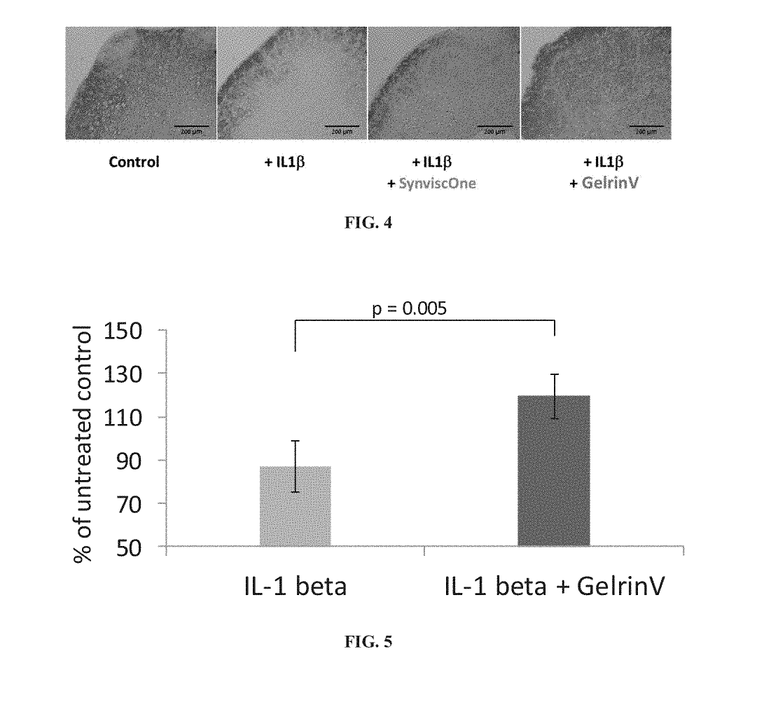

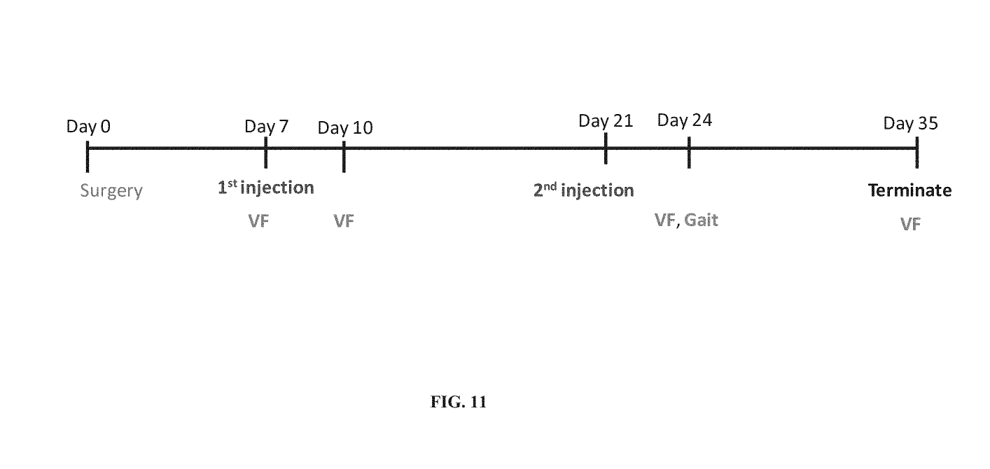

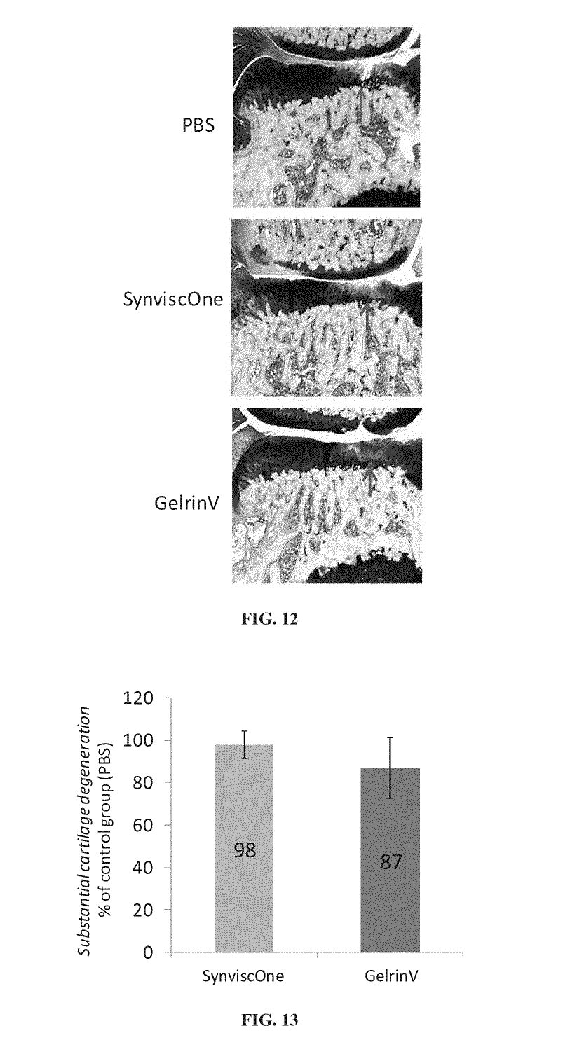

FIGS. 4-5 show that the conjugates protect cartilage in the presence of the pro-inflammatory cytokine IL-1β in an in vitro model. FIG. 4 further shows that a hyaluronic acid viscosupplement does not provide such protection. FIGS. 12-13 and 15-16B show that the conjugates protect cartilage against arthritis in vivo.

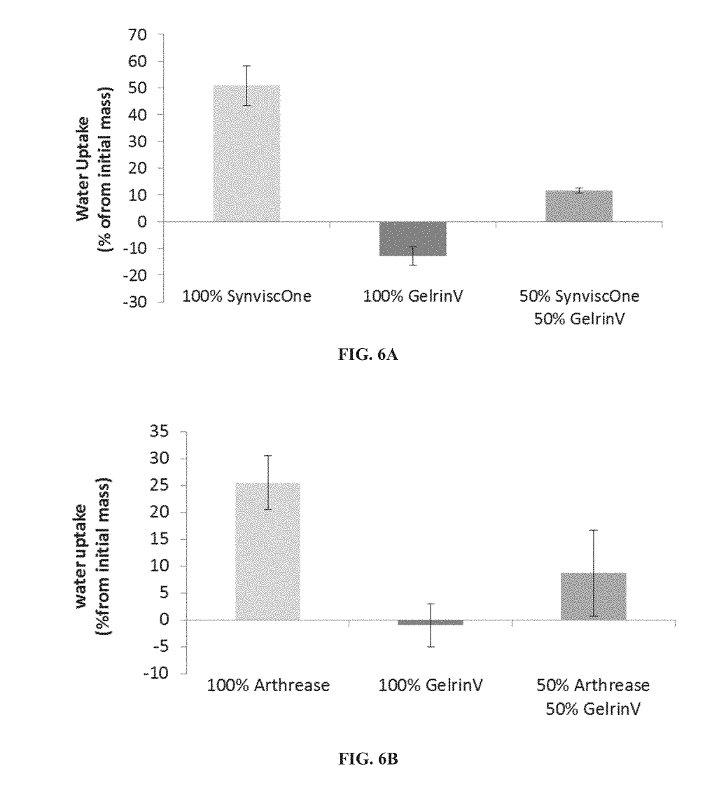

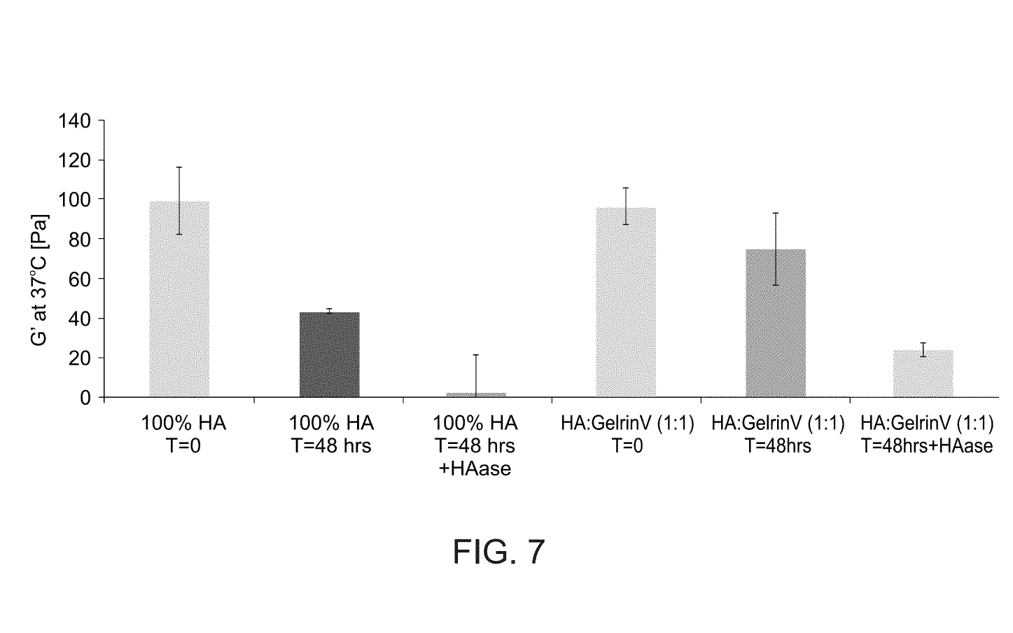

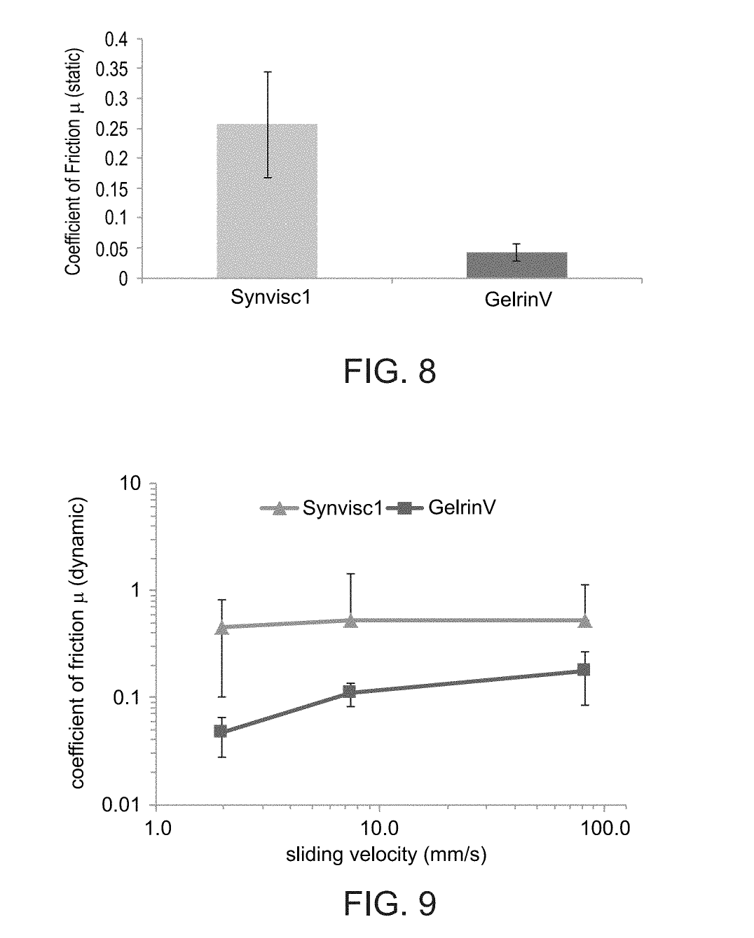

FIGS. 6A and 6B show that an exemplary composition comprising poloxamer-fibrinogen conjugates does not exhibit water uptake, in contrast to hyaluronic acid viscosupplements. FIG. 7 shows that viscosity of the composition comprising poloxamer-fibrinogen conjugates is longer lasting under physiological conditions than that of hyaluronic acid viscosupplements. FIGS. 8-9 show that the composition comprising poloxamer-fibrinogen conjugates is more lubricating than hyaluronic acid viscosupplements. FIGS. 15-16B show that hyaluronic acid viscosupplement does not exhibit the protective effect of the exemplary composition in vivo.

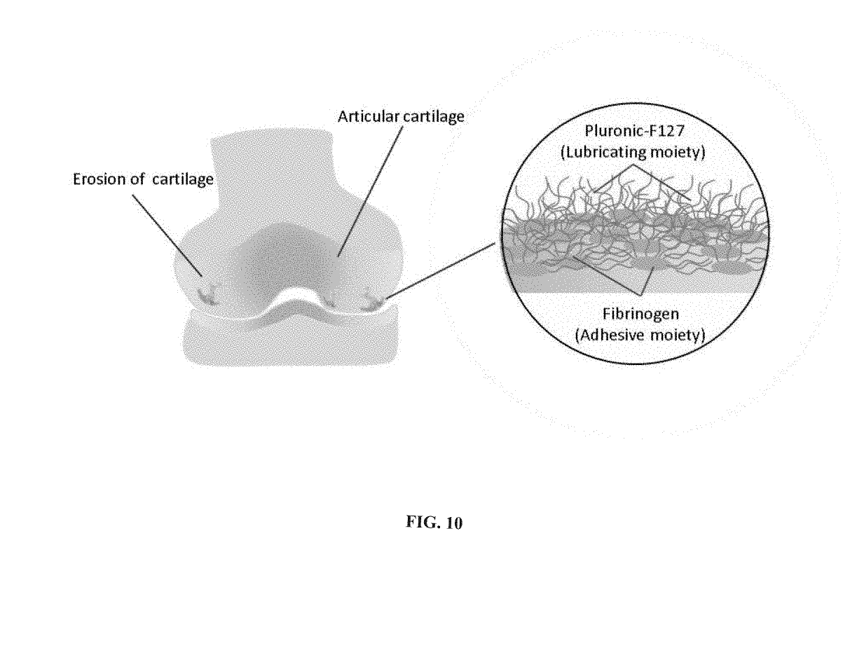

FIG. 10 shows a non-limiting mechanism by which a poloxamer-fibrinogen conjugate can adhere to the cartilage surface via the fibrinogen moiety and provide lubrication via the poloxamer moiety, according to optional embodiments of the invention.

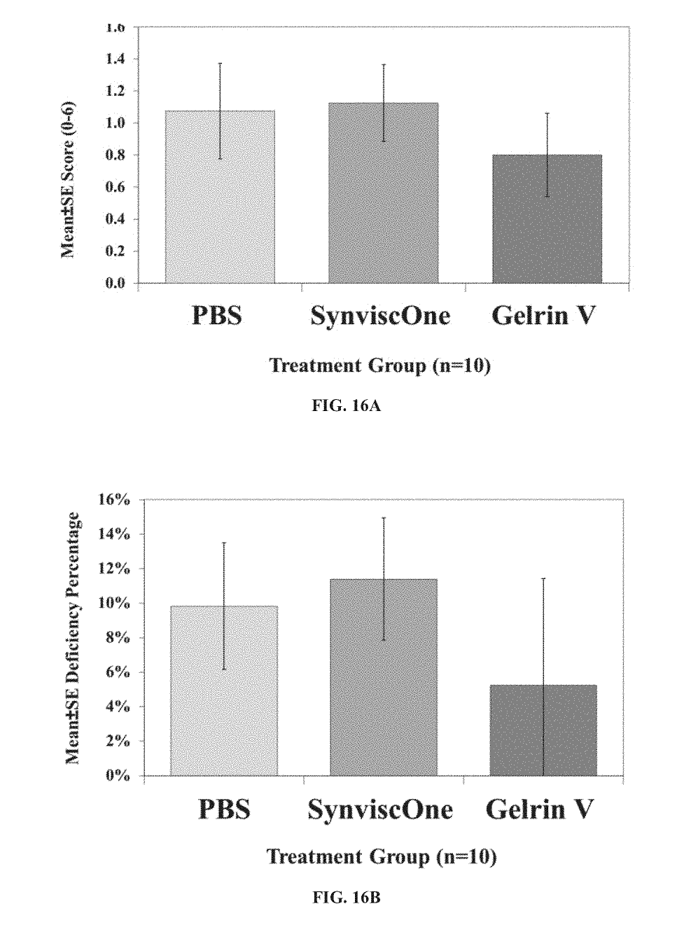

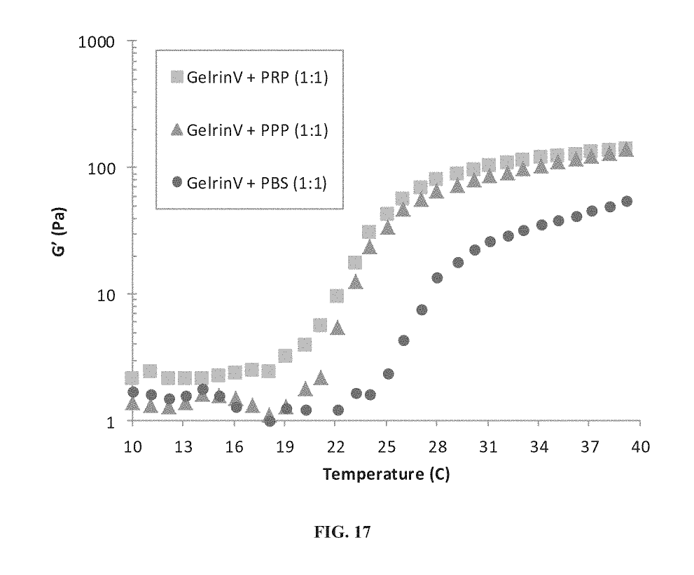

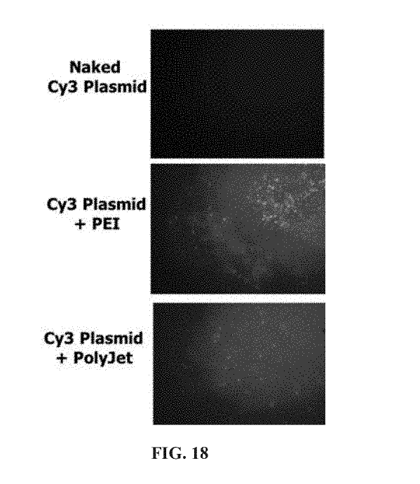

FIG. 17 shows that an exemplary composition comprising poloxamer-fibrinogen conjugates exhibits reverse thermal gelation when mixed with blood fractions. FIGS. 18-20 show that the composition effectively retains DNA-nanoplexes, thereby facilitating gene transfer to cells.

According to an aspect of some embodiments of the invention, there is provided a composition comprising a conjugate, the conjugate comprising a polypeptide having attached thereto at least two polymeric moieties. At least one of the polymeric moieties exhibits a reverse thermal gelation, as described herein according to any of the respective embodiments.

For brevity, a conjugate comprising a polypeptide having attached thereto at least two polymeric moieties (according to any of the respective embodiments described herein) is referred to herein interchangeably as a “polymer-protein conjugate” or simply as a “conjugate”.

In some embodiments of any of the embodiments described herein, the composition (according to any of the respective embodiments described herein) is for use in treating a condition as described herein.

In some embodiments of any of the embodiments described herein, the composition (according to any of the respective embodiments described herein) is for use in the manufacture of a medicament for use in treating a condition described herein.

According to an aspect of some embodiments of the invention, there is provided a method of treating a condition described herein, the method comprising administering the composition (according to any of the respective embodiments described herein) to a subject in need thereof, thereby treating the condition.

Polymer-Protein Conjugates:

The terms “polymer” and “polymeric” refer to a molecule or moiety composed primarily of a plurality of repeating units.

As mentioned hereinabove, at least one of the polymeric moieties attached to a polypeptide in a polymer-protein conjugate described herein exhibits a reverse thermal gelation.

In some embodiments of any of the embodiments described herein, at least two of the polymeric moieties attached to a polypeptide exhibit a reverse thermal gelation.

In some embodiments, each of the polymeric moieties attached to a polypeptide exhibit a reverse thermal gelation.

Herein, a polymeric moiety is considered to exhibit a reverse thermal gelation when an aqueous solution of a polymer which corresponds to the polymeric moiety (e.g., a polymer not attached to the abovementioned polypeptide) exhibits a reverse thermal gelation, as described herein.

As used herein, the phrase “reverse thermal gelation” describes a property whereby a substance (e.g., a composition or an aqueous solution of a polymer, according to any of the respective embodiments described herein) increases in viscosity upon an increase in temperature. The increase in viscosity may be, for example, conversion from a liquid state to a semisolid state (e.g., gel), conversion from a liquid state to a more viscous liquid state, or conversion from a semisolid state to a more rigid semisolid state. Herein, all such conversions are encompassed by the term “gelation”. The increase in temperature which effects gelation may be between any two temperatures. Optionally, the gelation is effected at a temperature within the range of 0° C. to 55° C.

Typically, reverse thermal gelation is mediated by the formation of non-covalent cross-linking (e.g., via hydrophobic interactions, ionic interactions, and/or hydrogen bonding) between molecules, wherein the degree of non-covalent cross-linking increases in response to an increase of temperature.

A variety of polymers exhibit a reverse thermal gelation. Each polymer may be characterized by a critical gelation temperature, wherein gelation is effected at the critical gelation temperature or at temperatures above the critical gelation temperature.

Herein, “critical gelation temperature” refers to the lowest temperature at which some gelation of a material is observed (e.g., by increase in shear storage modulus).

The polymeric moiety may be selected so as to impart to the conjugate containing same a reverse thermal gelation that is characterized by a critical gelation temperature within a temperature range (e.g., in a range of 0° C. to 55° C.) which allows for convenient manipulation of the properties of the conjugate and/or a composition comprising the conjugate, by exposure to an ambient temperature above and/or below the critical gelation temperature.

The critical gelation temperature of the polymer may be selected, for example, based on the intended use or desired properties of a conjugate. For example, the critical gelation temperature may be selected such that the conjugate is in a gelled state at a physiological temperature but not at room temperature, such that gelation may be effected in vivo. In another example, the critical gelation temperature may be selected such that the conjugate is in a gelled state at room temperature but not at a moderately lower temperature, such that gelation may be effected, for example, by removal from refrigeration.

The polymeric moiety optionally comprises a synthetic polymer. Poloxamers (e.g., F127 poloxamer) are exemplary polymers which exhibit a reverse thermal gelation at temperatures suitable for embodiments of the present invention.

The phrase “synthetic polymer” refers to any polymer which is made of a synthetic material, i.e., a non-natural, non-cellular material.

As used herein and in the art, a “poloxamer” refers to poly(ethylene oxide) (PEO)—poly(propylene oxide) (PPO) block copolymer having a PEO-PPO-PEO structure. Suitable poloxamers are commercially available, for example, as Pluronic® polymers.

The polymeric moiety may comprise one or more moieties which effect non-covalent cross-linking (e.g., hydrophobic moieties). The degree of gelation and the conditions (e.g., temperature) under which gelation is effected may optionally be controlled by the nature and the number of moieties which participate in non-covalent cross-linking.

The polymeric moiety may comprise from 1 and up to 100 and even 1000 moieties which participate in non-covalent cross-linking. In many embodiments, the higher the number of such moieties, and the larger the moieties are (e.g., the higher the molecular weights are), the lower the temperature under which gelation is effected.

The polymeric moiety may comprise one or more types of moieties which effect cross-linking. These moieties may effect non-covalent cross-linking via the same intermolecular interactions (e.g., hydrophobic interactions) or via different intermolecular interactions (e.g., hydrophobic and ionic interactions).

Polymers that exhibit reverse thermal gelation (also referred to in the art as RTG polymers) include, but are not limited to, poly(N-isopropylacrylamide), which undergoes reverse thermal gelation at temperatures above about 32-33° C., as well as copolymers thereof (e.g., poly(N-isopropylacrylamide-co-dimethyl-y-butyrolactone), poly(ethylene glycol)-poly(amino urethane) (PEG-PAU) block copolymers, poly(ε-caprolactone)-poly(ethylene glycol) (PCL-PEG) block copolymers (e.g., PCL-PEG-PCL), and poly(methyl 2-propionamidoacrylate). In addition, polyorganophosphazenes with PEG and hydrophobic oligopeptide side groups (which provide intermolecular hydrophobic interactions) have been described, which are gelled at temperatures of 35-43° C. [Seong et al., Polymer 2005, 46:5075-5081].

For example, a poloxamer moiety comprises a hydrophobic PPO moiety which mediates gelation. A polymeric moiety may optionally comprise one such PPO moiety, or alternatively, a plurality (e.g., 2, 3, 4, etc., up to 100 and even 1000 such moieties) of such moieties.

Similarly PCL-PEG copolymers comprise hydrophilic PEG and a relatively hydrophobic poly(ε-caprolactone) (PCL) moiety, and PEG-PAU copolymers comprise hydrophilic PEG and a hydrophobic poly(amino urethane) (PAU) moiety (e.g., a bis-1,4-(hydroxyethyl)piperazine-1,6-diisocyanato hexamethylene condensation polymer moiety).

Thus, in general, many block polymers exhibiting reverse thermal gelation may be prepared from a combination of hydrophilic and hydrophobic building blocks.

In some embodiments, each polymeric moiety comprises a poloxamer (e.g., F127 poloxamer).

Optionally, a polymeric moiety comprises one poloxamer.

Alternatively or additionally, at least one polymeric moiety comprises a plurality of poloxamer moieties. Polymers comprising a plurality of poloxamer moieties are commercially available, for example, as Tetronic® polymers.

According to optional embodiments, at least one of the polymeric moieties further comprises at least one cross-linking moiety capable of covalently said conjugate with a protein in vivo (e.g., under physiological conditions). Optionally, the polymeric moiety comprises from 1 to 10, optionally from 1 to 5, and optionally from 1 to 3 cross-linking moieties.

As used herein, the phrase “cross-linking moiety” refers to a moiety (e.g., a functional group in a polymeric moiety described herein) characterized by an ability to effect covalent cross-linking with a functional group of another molecule (e.g., a protein).

A conjugate according to some embodiments described herein may optionally be represented by the general formula:

X(—Y—Zm)n

wherein X is a polypeptide as described herein, Y is a polymeric moiety as described herein, Z is a cross-linking moiety as described herein, n is an integer greater than 1 (e.g., 2, 3, 4 and up to 20), and m represents the number of cross-linking moieties per polymeric moiety. Thus, m is 0 in embodiments lacking the optional cross-linking moiety, and m is 1 or an integer greater than 1, in embodiments which comprise the optional cross-linking moiety.

It is to be understood that as the above formula includes more than one —Y—Zm moiety, different —Y—Zm moieties in a conjugate may optionally have a different values for m.

Examples of suitable cross-linking moieties include, without limitation, an acrylate, a methacrylate, an acrylamide, a methacrylamide, and a vinyl sulfone, which are suitable for attachment to a thiol group (e.g., in a cysteine residue) via Michael-type addition; and an aldehyde and an N-hydroxysuccinimide, which are suitable for attachment to an amine group (e.g., in a lysine residue and/or N-terminus).

As exemplified in the Examples section herein, a polymeric moiety may comprise a plurality of such cross-linking moieties (e.g., acrylate), one of which attached the polymeric moiety to the polypeptide of the conjugate, and the remaining moieties being unbound to the polypeptide of the conjugate, and thus may optionally serve as cross-linking moieties.

Thus, in exemplary embodiments, the conjugate comprises poloxamer diacrylate (e.g., F127 poloxamer diacrylate) moieties, wherein one acrylate group in each moiety is attached to a cysteine residue of a polypeptide (e.g., denatured fibrinogen), and one acrylate group may optionally serve as a cross-linking moiety.

The polypeptide of the conjugate (according to any of the respective embodiments described herein) is at least 10 amino acids in length. In some embodiments of any of the embodiments described herein, the polypeptide is at least 20 amino acids in length, and optionally at least 50 amino acids in length.

The term “polypeptide” as used herein encompasses native polypeptides (either degradation products, synthetically synthesized polypeptides or recombinant polypeptides) and peptidomimetics (typically, synthetically synthesized polypeptides), as well as peptoids and semipeptoids which are polypeptide analogs, which may have, for example, modifications rendering the polypeptides more stable while in a body or more capable of penetrating into cells. Such modifications include, but are not limited to, N-terminus modification, C-terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the polypeptide may be substituted, for example, by N-methylated bonds (—N(CH₃)—CO—), ester bonds (—C(R)H—C—O—O—C(R)—N—), ketomethylene bonds (—CO—CH₂—), α-aza bonds (—NH—N(R)—CO—), wherein R is any alkyl, e.g., methyl, amine bonds (—CH₂—NH—), hydroxyethylene bonds (—CH(OH)—CH₂—), thioamide bonds (—CS—NH—), olefinic double bonds (—CH═CH—), retro amide bonds (—NH—CH—), peptide derivatives (—N(R)—CH₂—CO—), wherein R is the “normal” side chain, naturally presented on the carbon atom. These modifications can occur at any of the bonds along the polypeptide chain and even at several (2-3) at the same time.

As used herein throughout, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

According to some embodiments of any one of the embodiments described herein, the polypeptide comprises a protein or a fragment thereof.

In some embodiments, the terms “polypeptide” and “protein” are used interchangeably.

The protein may be a naturally occurring protein (e.g., a protein existing in eukaryotic and/or prokaryotic organisms, cells, cellular material, non-cellular material, and the like) or a polypeptide homologous (e.g., at least 90% homologous, optionally at least 95% homologous, and optionally at least 99% homologous) to a naturally occurring protein.

In some embodiments of any one of the embodiments described herein, the protein (or protein fragment) is denatured.

It is to be understood that the protein described herein may optionally comprise more than one polypeptide chain.

In embodiments comprising a protein characterized by more than one polypeptide chain, the conjugate described herein optionally comprises one polypeptide of the protein.

Alternatively, the conjugate described herein comprises a plurality of polypeptides of the protein (e.g., all of the polypeptides of the protein).

In some embodiments of any one of the embodiments described herein, the plurality of polypeptides are linked together (e.g., by non-covalent and/or covalent bonds) so as to form a multimer (e.g., a dimer, a trimer, a tetramer, a hexamer, etc.), the multimer having attached thereto at least two polymeric moieties, as described herein.

In some embodiments, the polypeptides of the protein are separate (e.g., separated by denaturation of the protein), such that the conjugate described herein is a mixture of different conjugate species, wherein each of the conjugate species comprises a different polypeptide.

In some embodiments of any one of the embodiments described herein, the polypeptide (e.g., protein or protein fragment) is selected so as to exhibit affinity to a biological substance. In some embodiments, the polypeptide is capable of adhering to cartilage.

In some embodiments of any one of the embodiments described herein, the polypeptide exhibits greater affinity to damaged cartilage than to undamaged cartilage.

In some embodiments, the polypeptide is capable of adhering to lubricin and/or hyaluronic acid. Fibronectin is a non-limiting example of such a polypeptide. Without being bound by any particular theory, it is believed that such adherence may contribute to lubrication Eguiluz et al. [Biomacromolecules 2015, 16:2884-2894].

Affinity to damaged cartilage and undamaged cartilage may be compared, for example, by contacting the polypeptide (e.g., per se or in the form of a conjugate described herein) with a cartilage surface comprising an abrasion, the cartilage being otherwise substantially undamaged, and comparing amounts of polypeptide adhering to than to the abraded and non-abraded portions of the surface (e.g., as exemplified herein).

Examples of proteins suitable for inclusion (per se or as fragments thereof) in conjugates described herein include, without limitation, a cell signaling protein, an extracellular matrix protein, a cell adhesion protein, a growth factor, albumin (e.g., serum albumin, for example, GenBank Accession No. NP_000468), von Willebrand factor (e.g., GenBank Accession No. NP_000543), protein A, a protease and a protease substrate. In some embodiments of any one of the embodiments described herein, the conjugate comprises an extracellular matrix protein.

Examples of extracellular matrix proteins include, but are not limited to, fibrinogen (e.g., α-chain—GenBank Accession No. NP_068657; β-chain—GenBank Accession No. P02675; γ-chain—GenBank Accession No. P02679), collagen (e.g., GenBank Accession No. NP_000079), fibronectin (e.g., GenBank Accession No. NP_002017), elastin, fibrillin, fibulin, laminin (e.g., GenBank Accession No. NP_000218) and gelatin.

Examples of cell signaling proteins include, but are not limited to, p38 mitogen-activated protein kinase (e.g., GenBank Accession No. NP_002736), nuclear factor kappaB (e.g., GenBank Accession No. NP_003989), Raf kinase inhibitor protein (RKIP) (e.g., GenBank Accession No. XP_497846), Raf-1 (e.g., GenBank Accession No. NP_002871), MEK (e.g., GenBank Accession No. NP_002746), protein kinase C (PKC) (e.g., GenBank Accession No. NP_002728), phosphoinositide-3-kinase gamma (e.g., GenBank Accession No. NP_002640), receptor tyrosine kinases such as insulin receptor (e.g., GenBank Accession No. NP_000199), heterotrimeric G-proteins (e.g., Galpha(i)—GenBank Accession No. NP_002060; Galpha(s)—GenBank Accession No. NP_000507; Galpha(q)—GenBank Accession No. NP_002063), caveolin-3 (e.g., GenBank Accession No. NP_001225), microtubule associated protein 1B, and 14-3-3 proteins (e.g., GenBank Accession No. NP_003397).

Examples of cell adhesion proteins include, but are not limited to, integrin (e.g., GenBank Accession No. NP_002202), intercellular adhesion molecule (ICAM) 1 (e.g., GenBank Accession No. NP_000192), N-CAM (e.g., GenBank Accession No. NP_000606), cadherin (e.g., GenBank Accession No. NP_004351), tenascin (e.g., GenBank Accession No. NP_061978), gicerin (e.g., GenBank Accession No. NP_006491), and nerve injury induced protein 2 (ninjurin2) (e.g., GenBank Accession No. NP_067606).

Examples of growth factors include, but are not limited to, epidermal growth factor (e.g., GenBank Accession No. NP_001954), transforming growth factor-β (e.g., GenBank Accession No. NP_000651), fibroblast growth factor-acidic (e.g., GenBank Accession No. NP_000791), fibroblast growth factor-basic (e.g., GenBank Accession No. NP_001997), erythropoietin (e.g., GenBank Accession No. NP_000790), thrombopoietin (e.g., GenBank Accession No. NP_000451), neurite outgrowth factor, hepatocyte growth factor (e.g., GenBank Accession No. NP_000592), insulin-like growth factor-I (e.g., GenBank Accession No. NP_000609), insulin-like growth factor-II (e.g., GenBank Accession No. NP_000603), interferon-γ (e.g., GenBank Accession No. NP_000610), and platelet-derived growth factor (e.g., GenBank Accession No. NP_079484).

Examples of proteases include, but are not limited to, pepsin (e.g., GenBank Accession No. NP_055039), low specificity chymotrypsin, high specificity chymotrypsin, trypsin (e.g., GenBank Accession No. NP_002760), carboxypeptidases (e.g., GenBank Accession No. NP_001859), aminopeptidases (e.g., GenBank Accession No. NP_001141), proline-endopeptidase (e.g. GenBank Accession No. NP_002717), Staphylococcus aureus V8 protease (e.g., GenBank Accession No. NP_374168), proteinase K (PK) (e.g., GenBank Accession No. P06873), aspartic protease (e.g., GenBank Accession No. NP_004842), serine proteases (e.g., GenBank Accession No. NP_624302), metalloproteases (e.g., GenBank Accession No. NP_787047), ADAMTS17 (e.g., GenBank Accession No. NP_620688), tryptase-γ (e.g., GenBank Accession No. NP_036599), matriptase-2 (e.g., GenBank Accession No. NP_694564).

Examples of protease substrates include the peptide or peptide sequences being the target of the protease protein. For example, lysine and arginine are the target for trypsin; tyrosine, phenylalanine and tryptophan are the target for chymotrypsin.

Such naturally occurring proteins can be obtained from any known supplier of molecular biology reagents.

According to some embodiments of any one of the embodiments described herein, the composition comprises a mixture of different conjugates, the different conjugates, for example, comprising different polypeptides.

In some embodiments, the composition comprises a mixture of conjugates, wherein at least one conjugate comprises albumin (e.g., serum albumin).

In some embodiments, the composition comprises a mixture of conjugates, wherein at least one conjugate comprises von Willebrand factor. In some embodiments, at least one conjugate comprises von Willebrand factor and at least one conjugate comprises albumin (e.g., serum albumin).

In some embodiments, the composition comprises a mixture of conjugates, wherein at least one conjugate comprises an extracellular matrix protein. In some embodiments, at least one conjugate comprises an extracellular matrix protein and at least one conjugate comprises albumin (e.g., serum albumin).

In some embodiments, at least one conjugate comprises an extracellular matrix protein and at least one conjugate comprises von Willebrand factor. In some embodiments, at least one conjugate comprises an extracellular matrix protein, at least one conjugate comprises albumin (e.g., serum albumin), and at least one conjugate comprises von Willebrand factor. In some of the aforementioned embodiments, the extracellular matrix protein comprises fibrinogen and/or fibronectin. In some of the aforementioned embodiments, the extracellular matrix protein comprises fibrinogen and fibronectin (in admixture).

According to some embodiments of any one of the embodiments described herein, the composition comprises at least one conjugate wherein the polypeptide comprises a fibrinogen polypeptide (α, β and/or γ chains of fibrinogen) or a fragment thereof. In some embodiments, the conjugate described herein comprises the α, β and γ chains of fibrinogen. In some embodiments, the polypeptide is a denatured fibrinogen (e.g., a mixture of denatured α, β and γ chains of fibrinogen).

Polymer-protein conjugates suitable for use in some of any of the embodiments of the invention are also described in International Patent Application Publication WO 2011/073991, the contents of which are incorporated herein by reference, especially contents describing polymer-protein conjugates.

Composition:

In some embodiments of any of the embodiments described herein, the composition comprises an aqueous solution of the conjugate.

Herein, the phrase “aqueous solution of the conjugate” refers to the conjugate being mixed with (e.g., dispersed and/or dissolved in) an aqueous medium, and is not to be understood as excluding compositions in which the conjugate is not dissolved or compositions having a high viscosity (e.g., in a form of a hydrogel).

In some embodiments of any of the embodiments described herein, a concentration of polymer-protein conjugates in the composition is at least 0.02 weight percent. In some embodiments, the concentration on conjugates is at least 0.05 weight percent. In some embodiments, the concentration is at least 0.1 weight percent. In some embodiments, the concentration is at least 0.2 weight percent. In some embodiments, the concentration is at least 0.5 weight percent. In some embodiments, the concentration is at least 1 weight percent. In some embodiments, the concentration is at least 1.5 weight percent. In some embodiments, the concentration is at least 2 weight percents. In some embodiments, the concentration is at least 2.5 weight percents.

In some embodiments of any of the embodiments described herein, a concentration of polymer-protein conjugates in the composition is no more than 20 weight percents. In some embodiments, the concentration of conjugates is no more than 10 weight percents. In some embodiments, the concentration is no more than 5 weight percents. In some embodiments, the concentration is no more than 2.5 weight percents.

In some embodiments of any of the embodiments described herein, a concentration of polymer-protein conjugates in the composition is in a range of from 0.02 to 20 weight percents. In some embodiments, the concentration of conjugates is in a range of from 0.1 to 10 weight percents. In some embodiments, the concentration of conjugates is in a range of from 0.5 to 5 weight percents. In some embodiments, the concentration of conjugates is in a range of from about 1 to about 2 weight percents.

In some embodiments of any of the embodiments described herein, the composition forms a gel at a temperature in a range of from 32° C. to 37° C., that is, at at least one temperature in the aforementioned range (optionally at each temperature in the aforementioned range), the composition is in a form of a gel. In some embodiments, the gel is a hydrogel, for example, wherein a composition comprising an aqueous solution of the conjugate (according to any of the respective embodiments described herein) forms a hydrogel at a temperature in a range of from 32° C. to 37° C.

As used herein and is well-known in the art, the term “hydrogel” refers to a material that comprises solid networks formed of water-soluble natural or synthetic polymer chains, often containing more than 99% water.

In some embodiments of any of the embodiments described herein, the gel (e.g., hydrogel) is characterized by a shear storage modulus of at least 15 Pa at 37° C. In some embodiments, the shear storage modulus is at least 50 Pa, optionally at least 100 Pa, and optionally at least 200 Pa, at 37° C.

As used herein and in the art, a “shear modulus” is defined as the ratio of shear stress to the shear strain. The shear modulus may be a complex variable, in which case the “storage modulus” is the real component and the “loss modulus” is the imaginary component. The storage modulus and loss modulus in viscoelastic solids measure the stored energy, representing the elastic portion, and the energy dissipated as heat, representing the viscous portion.

In some embodiments of any of the embodiments described herein, the composition is capable of undergoing reverse thermal gelation. In some embodiments, the composition is an aqueous solution according to any of the respective embodiments described herein.

In some embodiments of any of the embodiments described herein relating to a gel and/or hydrogel, the gel and/or hydrogel can be formed by reverse thermal gelation according to any of the respective embodiments described herein.

Optionally, the reverse thermal gelation of the composition occurs at a temperature below 55° C., optionally below 50° C., optionally below 40° C., and optionally below 30° C. Optionally, the reverse thermal gelation occurs at a temperature below about 32° C., such that at a physiological temperature in a range of about 32° C. (e.g., in extremities of the body) to 37° C., the composition is in a gelled state.

Optionally, the reverse thermal gelation of the composition occurs at a temperature above 0° C., optionally above 10° C., optionally above 20° C. and optionally above 30° C.

In some embodiments, the reverse thermal gelation of the composition occurs upon an increase of temperature from 0° C. to 55° C., optionally from 10° C. to 55° C., optionally from 10° C. to 40° C., optionally from 15° C. to 37° C., optionally from 20° C. to 37° C., and optionally from 20° C. to 32° C. Reverse thermal gelation which occurs upon an increase of temperature from a room temperature (e.g., about 20° C., about 25° C.) to a physiological temperature (e.g., about 32 to 37° C.) are particularly useful for some applications (e.g., medical applications), as gelation can be induced by transferring the composition from a room temperature environment to a physiological temperature, for example, by placing the composition in a body.

The temperature at which a composition undergoes reverse thermal gelation (according to any of the respective embodiments described herein) may optionally be controlled by varying the concentration of the conjugate in the composition.

Furthermore, the temperature at which a composition undergoes reverse thermal gelation (according to any of the respective embodiments described herein) may optionally be controlled by selecting a polymer with an appropriate gelation temperature for inclusion in the polymeric moiety, and/or by varying the concentration of polymeric moieties which exhibit reverse thermal gelation (e.g., by varying the number of polymeric moieties attached to a polypeptide and/or by varying the size of the polymeric moieties).

As exemplified in the Examples section, aqueous solutions comprising conjugates described herein may undergo reverse thermal gelation at relatively low concentrations, for example, less than 20 weight percents conjugate, optionally less than 10 weight percents, optionally less than 5 weight percents, and optionally less than 2 weight percents. Such low concentrations in a gel typically cannot be obtained using polymers (e.g., poloxamers) per se rather than polymer-protein conjugates described herein.

Without being bound by any particular theory, it is believed that the use of relatively low concentrations of conjugate is advantageous in that it can reduce undesirable interactions between the polymer and biomolecules in vivo, such as promotion of protein precipitation and/or irritation.

The reverse thermal gelation of a composition as described herein can be determined by measuring a shear storage modulus of the composition. A temperature-dependent increase in the storage modulus is indicative of a gel formation via a reverse thermal gelation.

In some embodiments of any of the embodiments described herein, the reverse thermal gelation according to any of the respective embodiments described herein increases a shear storage modulus (also referred to herein as “storage modulus”, or as G′) of the composition by at least ten-folds, optionally at least 30-folds, optionally at least 100-folds, and optionally at least 300-folds.

In some embodiments of any of the embodiments described herein, reverse thermal gelation according to any of the respective embodiments described herein increases a shear storage modulus of the aqueous solution to at least 15 Pa, optionally at least 20 Pa, optionally at least 50 Pa, optionally at least 100 Pa, and optionally at least 200 Pa.

In some embodiments of any of the embodiments described herein, the shear storage modulus of a composition according to any of the respective embodiments described herein before reverse thermal gelation (e.g., at a temperature below a temperature at which gelation occurs) is less than 2 Pa, optionally less than 1 Pa, optionally less than 0.5 Pa, and optionally less than 0.2 Pa.

In some embodiments of any of the embodiments described herein, the composition is an injectable composition, that is, it can be readily injected through a syringe needle (e.g., an 18-gauge needle).

Preferably, an injectable composition does not comprise particles large enough to clog a needle, and has a sufficiently low viscosity to allow injection. Such low viscosity may be, for example, a relatively low viscosity of a composition prior to reverse thermal gelation (e.g., according to any of the respective embodiments described herein) and/or a relatively low viscosity obtained upon application of shear stress during injection (e.g., a thixotropic composition).

In some embodiments of any of the embodiments described herein, the composition is substantially devoid of covalent cross-linking between polymer-protein conjugates.

Without being bound by any particular theory, it is believed that considerable covalent cross-linking of the conjugates may result in excessive rigidity of the composition, which could limit the ability of the composition to adjust to the changing geometry in a moving joint.

In some embodiments of any of the embodiments described herein, the composition is biodegradable. For example, a gel (e.g., hydrogel) according to any of the respective embodiments described herein is optionally a biodegradable gel, i.e., the gel degrades in contact with a tissue and/or a cell (e.g., by proteolysis and/or hydrolysis).

In some embodiments of any of the embodiments described herein, the composition (e.g., a gel according to any of the respective embodiments described herein) is characterized by little or no water uptake upon incubation with an aqueous liquid. In some embodiments, composition is characterized by water uptake of less than 20 weight percents upon incubation with an aqueous liquid for 48 hours at a temperature of 37° C. In some embodiments, the water uptake is less than 15 weight percents upon incubation for 48 hours at 37° C. In some embodiments, the water uptake is less than 10 weight percents upon incubation for 48 hours at 37° C. In some embodiments, the water uptake is less than 5 weight percents upon incubation for 48 hours at 37° C. In some embodiments, the water uptake is less than 2 weight percents upon incubation for 48 hours at 37° C. In some embodiments, the water uptake is less than 1 weight percent upon incubation for 48 hours at 37° C.

Herein, the phrase “water uptake” refers to the weight ratio of net increase in amount of water in the composition to initial weigh of composition.

Water uptake by a composition may optionally be determined by incubating an amount (e.g., 0.3 ml) of a composition with an amount (e.g., 1 ml) aqueous liquid such as phosphate buffer saline (e.g., pH 7.4) under the indicated conditions, and comparing the weight of the composition before and after incubation, with the change in weight being assumed to represent water uptake (e.g., as exemplified in the Examples section herein).

Without being bound by any particular theory it is believed that compositions with reduced water uptake tend to be more resistant to loss of beneficial activity via dilution of the composition in vivo.

In some embodiments of any of the embodiments described herein, the composition comprises at least one additional therapeutically active agent, i.e., a therapeutically active agent in addition to the conjugate described herein.

In some embodiments of any of the embodiments described herein, the composition comprising at least one additional therapeutically active agent forms a hydrogel at a temperature in a range of from 32° C. to 37° C. (according to any of the respective embodiments described herein). In some embodiments, the composition is an aqueous composition (according to any of the respective embodiments described herein).

Examples of additional therapeutically active agents which may be included in some embodiments described herein include, without limitation, a hyaluronic acid, an anti-inflammatory agent, an analgesic, a growth factor, a blood fraction (e.g., an autologous blood fraction), a nucleic acid, and a cell (preferably live cells). Hyaluronic acid, blood fractions, and nucleic acid are exemplary additional therapeutically active agents.

Examples of suitable growth factors include, without limitation, TGF-β (e.g., TGF-β1), insulin-like growth factors (e.g., IGF-1), fibroblast growth factors (e.g., FGF-2), bone morphogenetic proteins (e.g., BMP-2, BMP-7) and growth/differentiation factors (e.g., GDF-5), as well as any other growth factors described herein.

Examples of suitable anti-inflammatory agents include, without limitation, etanercept, infliximab, adalimubab, IL-1Ra, interferon-β, NSAIDs, and corticosteroids.

Examples of suitable analgesics include, without limitation, lidocaine, bupivacaine, ropivacaine, opiates, and botulinum toxin A.

In embodiments comprising a blood fraction in an aqueous composition, the blood fraction may optionally provide substantially all of the water in the aqueous composition. Alternatively, water present in the blood fraction is supplemented with water from an additional source, such as an aqueous carrier included in the composition.

In some embodiments of any of the embodiments described herein, at least 20 weight percents of the composition is one or more blood fractions. In some embodiments, at least 30 weight percents of the composition is the blood fraction(s).

In some embodiments, at least 40 weight percents of the composition is the blood fraction(s). In some embodiments, at least 50 weight percents of the composition is the blood fraction(s). In some embodiments, at least 60 weight percents of the composition is the blood fraction(s). In some embodiments, at least 70 weight percents of the composition is the blood fraction(s). In some embodiments, at least 80 weight percents of the composition is the blood fraction(s). In some embodiments, at least 90 weight percents of the composition is the blood fraction(s). In some embodiments, the composition consists essentially of the conjugate (according to any of the respective embodiments described herein) in combination with one or more blood fraction.

Examples of blood fractions suitable for inclusion in compositions described herein include, without limitation, platelet-rich plasma and platelet-poor plasma.

In some embodiments, the blood fractions are autologous blood fractions, and in some embodiments, the autologous blood fractions include platelet-rich plasma.

Hyaluronic acid (HA), also called hyaluronate or hyaluronan, is a high molecular weight non-sulfated glycosaminoglycan (GAG) present in all mammals. HA is composed of repeating disaccharide units composed of (β-1,4)-linked D-glucuronic acid and (β-1,3)-linked N-acetyl-D-glucosamine.

Herein, the term “hyaluronic acid” encompasses low and high molecular weight hyaluronic acid, in its pure (acid) or salt form, as well as all cross-linked, modified or hybrid forms of hyaluronic acid.

Cross-linker agents for forming cross-linked hyaluronic acid include, without limitation, glutaraldehyde and other aldehydes, dialdehydes, genipin, cinnamic acid or derivatives of it, synthetic cross-linkers from the carbodiimide family (EDC), divinylsulfone, BODE and mannitol, ribose and other sugars.

Examples of modified hyaluronic acid and modified groups which may be present in modified hyaluronic acid include, without limitation, polyvinylpyrrolidone-sodium hyaluronate, disulfide cross-linked modified hyaluronic acid, glycidyl trimethylammonium chloride (GTAC), phenyl succinic acid modified hyaluronic acid derivatives, sodium caproyl hyaluronate, sodium tyramino-hyaluronate, sodium rhodaminylamino-hyaluronate, sodium fluoresceinylamino-hyaluronate, DTPA-hyaluronate, DTPA (Gd)-hyaluronate, sodium formyl hyaluronate, sodium palmitoyl hyaluronate, sodium propinylamino-hyaluronate, sodium azidopropylamino-hyaluronate.

Examples of hybrid modified hyaluronic acid includes, without limitation, diphenylalanine hyaluronic acid, albumin hyaluronic acid, fibrinogen or fibrin hyaluronic acid, chitosan hyaluronic acid and any other kind protein or carbohydrate polymers with hyaluronic acid.

Optionally, the hyaluronic acid is in a form of a commercially available composition such as an aqueous solution or gel (e.g., viscosupplement), for example,

Synvisc-One® or Arthrease® viscosupplements. In embodiments comprising a hyaluronic acid composition (e.g., viscosupplement), the hyaluronic acid composition may optionally provide a portion or even substantially all of the water in the aqueous composition.

Examples of suitable nucleic acids (e.g., DNA) include, without limitation, gene vectors (e.g., plasmids, cosmids, artificial chromosomes, and/or viral vectors), antisense nucleic acids, siRNA, shRNA, micro-RNA, ribozymes and DNAzymes.

The term “siRNA” refers to small inhibitory RNA duplexes (generally between 18-30 base-pairs) that induce the RNA interference (RNAi) pathway. Typically, siRNAs are chemically synthesized as 2lmers with a central 19 bp duplex region and symmetric 2-base 3′-overhangs on the termini, although it has been recently described that chemically synthesized RNA duplexes of 25-30 base length can have as much as a 100-fold increase in potency compared with 2lmers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

miRNAs may direct an RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

DNAzymes are single-stranded polynucleotides which are capable of cleaving both single and double stranded target sequences [Breaker, R. R. and Joyce, G. Chemistry and Biology 1995; 2:655; Santoro, S. W. & Joyce, G. F. Proc. Natl, Acad. Sci. USA 1997; 943:4262] A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions [for review of DNAzymes see Khachigian, Curr Opin Mol Ther 4:119-21 (2002)]. Examples of construction and amplification of synthetic, engineered DNAzymes recognizing single and double-stranded target cleavage sites have been disclosed in U.S. Pat. No. 6,326,174.

Ribozymes are another molecule capable of specifically cleaving an mRNA transcript, and are increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms, have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Further details regarding construction and uses of nucleic acids according to some embodiments of the invention are described herein.

It is expected that during the life of a patent maturing from this application many relevant therapeutically active agents will be developed and the scope of the term “therapeutically active agent” is intended to include all such new technologies a priori.

In some embodiments of any of the embodiments described herein, the composition is capable of sustained release of said therapeutically active agent (e.g., under physiological conditions, such as an aqueous environment at 37° C. and pH 7.4), that is, the therapeutically active agent can be released gradually from the composition over a prolonged period of time (e.g., at least 24 hours).

In some embodiments, sustained release is characterized by retention of at least 20% of the therapeutically active agent upon incubation of the composition (e.g., 0.3 ml) for 48 hours in an aqueous environment (e.g., at 37° C. and pH 7.4), e.g., as exemplified in the Examples section herein. In some embodiments, the retention of the therapeutically active agent upon incubation for 48 hours is at least 30%, optionally at least 40%, optionally at least 50%, optionally at least 60%, optionally at least 70%, optionally at least 80%, and optionally at least 90%. Typically, the aqueous environment has a considerably larger volume than the composition such that re-entry of previously released therapeutically active agent into the composition from the environment is minimal. Quantification of the amount of therapeutically active agent may be performed by any suitable technique known in the art.

Applications:

In some embodiments of any of the embodiments described herein relating to use of the composition (according to any of the respective embodiments described herein) for treating a condition, the condition is associated with degeneration of articular cartilage and/or with subchondral bone loss.

The treating according to some of any of the embodiments described herein comprises intra-articular administration of the composition, for example, by intra-articular injection.

Herein, the term “intra-articular” refers to administration and/or injection into a joint, and encompasses administration into any tissue and/or space in the joint, including into cartilage, bone, and/or synovial cavity.

Intra-articular injection may optionally be effected by administering a composition sufficiently fluid to be injectable. Such a composition may be relatively fluid (non-viscous) in general, or the composition may become less fluid (e.g., undergo gelation) following administration, for example, upon being subjected to a physiological temperature. Non-limiting examples of such compositions include compositions which exhibit reverse thermal gelation (according to any of the respective embodiments described herein), which undergo gelation at physiological temperatures (e.g., in a range of from 32 to 37° C.) and which may be administered at a lower than physiological temperature at which the composition is relatively fluid (e.g., in a range of from 4 to 20° C.).

In some embodiments of any of the embodiments described herein, at least a portion of the articular cartilage subject to degeneration is in a synovial joint.

In some embodiments of any of the embodiments described herein, a condition associated with degeneration of articular cartilage is associated with friction at a surface of the articular cartilage. In some such embodiments, the composition is characterized by a static coefficient of friction which is less than 0.2. In some embodiments, the static coefficient of friction is less than 0.15. In some embodiments, the static coefficient of friction is less than 0.1. In some embodiments, the static coefficient of friction is less than 0.05.

Without being bound by any particular theory, it is believed that compositions characterized by relatively low coefficients of friction are effective at lubricating articular cartilage, thereby benefiting a subject afflicted by articular cartilage friction.

Coefficient of friction measurements may optionally be performed according to procedures known in the art (e.g., as described by Singh et al. [Nat Mater 2014, 13:988-995]). For example, a tested composition may optionally be placed between two surfaces (e.g., polytetrafluoroethylene surfaces) with an applied normal force (e.g., 0.01-0.02 N) and torque, as exemplified herein in the Examples section. A static friction coefficient (μₛ) can thus be determined using the equation: μₛ=τₘₐₓ/(R_{eff}*N), wherein τₘₐₓ is the maximal torque value (e.g., during the startup period of the test), R_{eff} is the effective radius of the surface to which the torque is applied, and N is the normal force.

Osteoarthritis is a non-limiting example of a condition wherein degeneration of articular cartilage is associated with friction at a surface of the articular cartilage.

In some embodiments of any of the embodiments described herein, degeneration of articular cartilage is associated with an inflammation, for example, wherein the inflammation induces cartilage degeneration. I some such embodiments, the composition for administration (according to any of the respective embodiments described herein) is capable of reducing degeneration of cartilage induced by inflammation.

Arthritis is a non-limiting example of a condition associated with degeneration of articular cartilage, wherein the degeneration is associated with an inflammation.

Herein and in the art, the term “arthritis” refers to a joint disorder that involves inflammation, and encompasses, without limitation, osteoarthritis, rheumatoid arthritis, psoriatic arthritis, septic arthritis, gout, pseudo-gout, ankylosing spondylitis, juvenile idiopathic arthritis, Still's disease, and arthritis secondary to lupus erythematosus.

In some embodiments of any of the embodiments described herein, the condition is associated with a subchondral bone cyst. In some embodiments, the condition is characterized by joint pain, optionally in the absence of observable damage to cartilage.

Osteoarthritis is a non-limiting example of a condition associated with a subchondral bone cyst. Treatment of osteoarthritis may optionally be prophylactic, e.g., wherein a subject with a subchondral bone cyst is identified as being at risk for osteoarthritis, but has not been diagnosed with osteoarthritis.

In some embodiments of any of the embodiments described herein relating to a bone cyst, treatment is effected by placing the composition in the bone cyst, for example, by injecting the composition into the bone cyst. In some embodiments, the composition forms a gel (according to any of the respective embodiments described herein) in situ (in the cyst)

Injection into hard tissue, such as cartilage and/or bone, may optionally be effecting by any suitable technique known in the art, for example, comprising drilling into the cartilage and/or bone. Suitable techniques include, for example, procedures and apparatuses described in U.S. Patent Application Publication 2011/0125156, the contents of which are incorporated herein by reference (especially contents describing administration of a composition into a subchondral bone defect); and/or marketed under the name Subchondroplasty™.

In some embodiments of any of the embodiments described herein, a composition according to any of the respective embodiments described herein is capable of reducing pain severity following injection into a subchondral bone cyst.

In some embodiments of any of the embodiments described herein, a composition according to any of the respective embodiments described herein is selected capable of enhancing subchondral bone reconstitution following injection into a subchondral bone cyst.

Without being bound by any particular theory, it is believed that subchondral bone reconstitution in a region of a subchondral bone cyst may lower a risk and/or severity of osteoarthritis in a subject following treatment.

It is further believed that a composition placed within a bone (e.g., bone cyst) according to any of the respective embodiments described herein advantageously allows continued transmission of nutrients and/or oxygen through the bone volume occupied by the composition (e.g., due to a porous nature of a hydrogel), while also facilitating invasion of the bone volume by cells (e.g., thereby repairing a bone cyst).

In contrast, alternative compositions and/or bone cements which merely fill a bone volume with a mineral substance such as calcium phosphate, or with a polymer such as poly(methyl methacrylate), may be less amenable to transmission of nutrients and/or oxygen.

In some embodiments of any of the embodiments described herein relating to a composition comprising an additional therapeutically active agent, the composition is for use in treating a condition treatable by the therapeutically active agent. In some such embodiments, the condition is treatable by local administration of the therapeutically active agent, and the aforementioned treating comprises local administration of the composition (to a region of the body in which local administration of the therapeutically active agent is beneficial).

A blood fraction (according to any of the respective embodiments described herein) is a non-limiting example of an additional therapeutically active agent which may be included in a composition (e.g., according to any of the respective embodiments described herein) for treating arthritis (e.g., osteoarthritis), nerve injury, tendinitis (e.g., chronic tendinitis), muscle injury (e.g., cardiac muscle injury), bone injury (e.g., bone cyst), and/or surgical injury (e.g., an incision site). In some such embodiments, the blood fraction is a platelet-rich plasma.

Hyaluronic acid is a non-limiting example of an additional therapeutically active agent which may be included in a composition (e.g., according to any of the respective embodiments described herein) for treating arthritis, for example, osteoarthritis.

As exemplified herein, incorporation of hyaluronic acid (including cross-linked or non-cross-linked hyaluronic acid) in a composition such as described herein may reduce dilution and/or clearance of hyaluronic acid from an intended location in a physiological environment, for example, an arthritic joint.

The use of hyaluronic acid is known in the art to be limited (inter alia) by its rapid in vivo enzymatic digestion by a family of enzymes called hyaluronidases [Jiang et al, Physiol Rev 2011, 91:221-264; and Girish & Kemparaju, Life Sciences 2007, 80:1921-1943], which limits its longevity in vivo. This enzymatic degradation results in a loss of hyaluronic acid effect within short time after its application, and, in addition, the short segments of the degraded HA have been suggested to play a role in inducing local inflammation.

As further exemplified herein, incorporation of hyaluronic acid in a composition such as described herein may protect hyaluronic acid from degradation by hyaluronidase.

In some embodiments of any of the embodiments described herein relating to use of a cell, the condition is treatable by a substance produced by said cell.

Examples of suitable therapeutically active substances which may be produced by a cell include, without limitation, polypeptides (including naturally occurring proteins and artificial polypeptide sequences) such as growth factors (e.g., TGF-β, insulin-like growth factors, fibroblast growth factors, bone morphogenetic proteins and growth/differentiation factors) and anti-inflammatory polypeptides (e.g., etanercept, infliximab, adalimubab, IL-1Ra, interferon-β); polysaccharides (e.g., hyaluronic acid); and nucleic acids (e.g., antisense nucleic acid, siRNA), optionally for downregulating a pro-inflammatory protein. Techniques regarding therapeutically active substances produced by cells are described, for example, by Madry et al. [Cartilage 2011, 2:201-225], Madry & Cucchiarini [J Gene Med 2013, 15:343-355] and Evans et al. [Transl Res 2013, 161:205-2016].

In some embodiments of any of the embodiments described herein relating to use of a composition comprising a nucleic acid, the use comprises delivery of a gene comprised by the nucleic acid to cells. In some embodiments, the use is for treating a condition treatable by expression of the gene in vivo, for example, by a protein encoded by the gene.

According to an aspect of some embodiments of the invention, there is provided a method of effecting gene delivery, the method comprising contacting at least one cell with a composition comprising a conjugate and a nucleic acid (according to any of the respective embodiments described herein), wherein the nucleic acid comprising the gene for delivery. The method may optionally be effected in vivo or ex vivo.

In some embodiments according to this aspect, the at least one cell is encapsulated by the composition and/or cultured on a surface of the composition, for example, wherein the method is effected ex vivo.

In some embodiments according to any of the embodiments relating to nucleic acid and/or gene delivery (according to any of the aspects described herein), a nucleic acid construct (also referred to herein as an “expression vector”) includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed.

Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMnco-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of polypeptides (e.g., a polypeptide according to any of the respective embodiments described herein) since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of some embodiments of the invention can also include sequences engineered to enhance stability, production, purification, yield or toxicity of the expressed peptide. For example, the expression of a fusion protein or a cleavable fusion protein comprising the polypeptide of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the polypeptide and the heterologous protein, the polypeptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned hereinabove, a variety of prokaryotic or eukaryotic cells can be used as host-expression systems to express the polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, N.Y., Section VIII, pp 421-463.

Other expression systems such as insects and mammalian host cell systems which are well known in the art and are further described hereinbelow can also be used by some embodiments of the invention.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

As used herein a “pharmaceutical composition” refers to a preparation of one or more of the active ingredients described herein with other chemical components such as pharmaceutically acceptable carriers and excipients. The purpose of a pharmaceutical composition is to facilitate administration of a compound to an organism.

Herein the term “active ingredient” refers to a polymer-protein conjugate and/or to an additional therapeutically active agent (according to any of the respective embodiments described herein).

Hereinafter, the phrase “pharmaceutically acceptable carrier” refers to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Regimens for combination of the pharmaceutical composition of the invention with additional agents can be formulated according to parameters such as specific conditions or diseases, health status of the subject, methods and dose of administration, and the like. Determination of such combination regimen can be done, for example, by professionals such as attending physicians, hospital staff, and also according to predetermined protocols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

The pharmaceutical compositions of the invention may optionally include a “therapeutically effective amount” of an active agent according to any of the respective embodiments described herein. A “therapeutically effective amount” refers to an amount effective, at dosages and for periods of time necessary, to achieve the desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of the active agent are outweighed by the therapeutically beneficial effects.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that any dosage ranges set forth herein are exemplary only and are not intended to limit the scope or practice of the claimed composition.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more pharmaceutically acceptable carriers comprising excipients and auxiliaries, which facilitate processing of the ingredients of the composition described herein into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art, for example, surfactants.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose; and/or pharmaceutically acceptable polymers such as polyvinyl pyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes.

Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use, as detailed hereinabove.

The pharmaceutical composition of some embodiments of the invention may also be formulated in rectal compositions such as suppositories or retention enemas, using, e.g., conventional suppository bases such as cocoa butter or other glycerides.

As discussed herein, the pharmaceutical composition may optionally be administered in a local rather than systemic manner, for example, via injection of the pharmaceutical composition directly into a tissue region (e.g., a joint) of a patient or other subject in need thereof.

Herein, the term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions suitable for use in context of some embodiments of the invention include compositions wherein the active ingredients are contained in an amount effective to achieve the intended purpose. More specifically, a therapeutically effective amount means an amount of active ingredients (modified DNase I according to any of the respective embodiments described herein) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose (of conjugate described herein and/or an additional therapeutically active agent described herein) can be estimated initially from in vitro and cell culture assays, and in animal models. For example, a dose can be formulated in animal models (e.g., according to procedures described herein) to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in humans.

The dosage (of conjugate described herein and/or an additional therapeutically active agent described herein) may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually, for example, to provide levels of the conjugate described herein and/or an additional therapeutically active agent described herein in cells, serum, and/or joint which are sufficient to induce or suppress the biological effect (e.g., minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from a single administration to a plurality of administrations over the course of several days or up to several years or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions according to any of the respective embodiments of the invention described herein may also be prepared, placed in an appropriate container, and labeled for treatment of an indicated condition, as is further detailed herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

Antibodies (rabbit anti-collagen II, ab34712, and mouse anti-human fibrin, ab58207) were obtained from Abcam.

Green fluorescent protein plasmids (pmax-GFP) were obtained from Amaxa.

F127 poloxamer (Kolliphor® P407), having a molecular weight of 12.6 kDa, was obtained from BASF.

F127 poloxamer-diacrylate (F127-DA) was prepared by acrylation of F127 poloxamer according to procedures described in International Patent Application Publication WO 2011/073991.

Fibrinogen (human; Tissee™) was obtained from Baxter.

PolyJet™ transfection agent was obtained from SignaGen.

PEI (polyethylenimine) transfection reagent (25 kDa, linear) was obtained from University of Uppsala, Sweden.

Tris(2-carboxyethyl)phosphine hydrochloride was obtained from Sigma.

Cell Propagation:

Primary ovine chondrocytes were thawed and seeded in monolayer and cultured to confluence in the presence of chondrocyte standard medium (high glucose DMEM, 10% fetal bovine serum, 100 units/ml penicillin/streptomycin, non-essential amino-acids, ascorbic acid). Passage 2-6 monolayer chondrocytes were harvested for experiments.

C2C12 myoblast cells were passaged using growth medium (high glucose Dulbecco's modified Eagle medium supplemented with 10% fetal bovine serum and 2.5% HEPES, pH 7.4, and antibiotics (penicillin/streptomycin). Before each gene delivery experiment, cells were grown for 24 hours on plates in growth medium at 100% confluence, then trypsinized, centrifuged and collected in 15 ml tubes. Cells were used up to passage 11.

Conjugation of F127 Poloxamer Diacrylate (F127-DA) to Fibrinogen

F127-DA was conjugated to fibrinogen to obtain a solution of F127-fibrinogen conjugate (also referred to herein interchangeably as “GelrinV”) using a modification of the procedure described in International Patent Application Publication WO 2011/073991).

A 9.26 mg/ml solution of human fibrinogen in 150 mM phosphate buffer saline (PBS) with 8 M urea was supplemented with tris(2-carboxyethyl) phosphine hydrochloride (TCEP HCl) at a molar ratio of 1.5:1 TCEP HCl to fibrinogen cysteines. After dissolution, the pH of the solution was adjusted to 8.0 using 1 M NaOH. F127-DA in a solution of PBS and 8 M urea (146.7 mg/ml) was added and reacted for 3 hours at room temperature. The molar ratio of synthetic polymer to fibrinogen cysteines was 1:1. After 3 hours the reaction solution was transferred to a dialysis tube with a 12-14 kDa cutoff (CelluSep) and dialyzed against PBS (pH 7.4) at 4° C.) in order to remove the urea. The net fibrinogen concentration was determined using a standard BCA™ Protein Assay (Pierce Biotechnology) and the relative amounts of total conjugated product (dry weight) to fibrinogen content (BCA values) were compared.

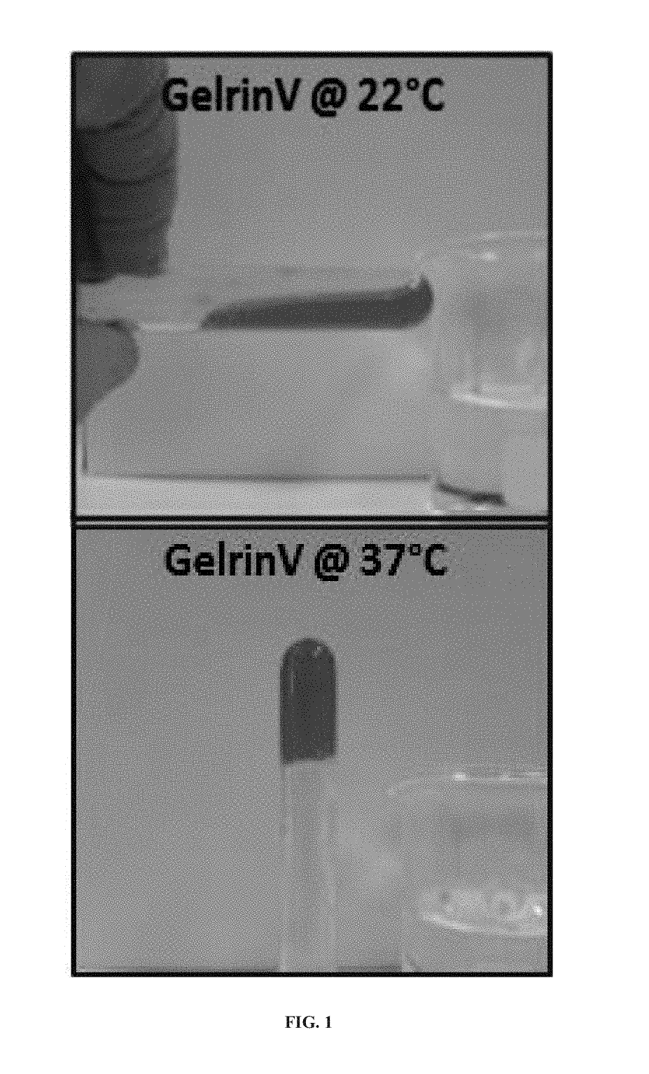

As shown in FIG. 1, the GelrinV behaved as a gel at a physiological temperature (37° C.) and as a viscous liquid (which could readily be injected through a thin needle) at room temperature (22° C.).

Florescent Labeling of F127-Fibrinogen (GelrinV):

6 ml of F127-fibrinogen solution (GelrinV) was placed in a dialysis tube (CelluSep) with a 12-14 kDa cutoff, and inserted into a PBS solution (pH 7.4) containing 0.025 mg/ml NHS-FITC (N-hydroxysuccinimide-fluorescein isothiocyanate; Thermo Scientific) for 8 hours at room temperature. After labeling the fibrinogen amine groups, the dialysis tube was inserted into a 4,000 ml PBS (phosphate buffer saline) solution to remove free NHS-FITC molecules from GelrinV.

Shear Storage Modulus (G′) Measurements:

Temperature-controlled rheological measurements were carried out using an AR-G2 rheometer (TA Instruments) equipped with a Peltier plate temperature-controlled base. 20 mm stainless steel plate geometry was used in all experiments. Each measurement was carried out with 0.2 ml sample. The testing conditions for the rheological measurements were 2% strain at an oscillation frequency of 2.5 Hz.

Coefficient of Friction Measurements:

Coefficient of friction (CoF; μ) measurements were performed according to procedures described by Singh et al. [Nat Mater 2014, 13:988-995]. Using an AR-G2 rheometer (TA Instruments) equipped with a Peltier plate temperature-controlled base, 0.5 ml of test item was placed on a flat polytetrafluoroethylene mold stage (25 mm in diameter). A polytetrafluoroethylene ring (annular geometry, 15 mm outer diameter and 9 mm inner diameter) which was attached to an upper, 20 mm stainless steel geometry was lowered until a normal force of 0.01-0.02 N was applied. During each test, torque (τ) and normal force (N) were measured, and instantaneous measurements of μₖ, the kinetic friction coefficient, were determined using the following equation: μₖ=τ/(R_{eff}*N). Static friction coefficients were determined using the equation: μₛ=τₘₐₓ/R_{eff}*N) at the maximal torque value found during the startup period of the test. The effective radius (R_{eff}) of the annulus geometry used for the calculations was 13.1 mm.

Allodynia Evaluation:

Mechanical allodynia (pain due to a stimulus that does not normally provoke pain) was evaluated using the von Frey method, based on the response of rats to the application of calibrated filaments (Bioseb, France) to the foot. Filaments were identified by a number representing log 10 of the force in mg×10. Rats were habituated to a testing rack three times (45-60 minutes) prior to baseline evaluation. Testing began with three applications of the 4.31 filament to both left and right hind paws. A response was recorded when the rat had an obvious reaction to the pressure of the hair, typically manifested as lifting of the hind paw from the grate to relieve the pressure. Three applications were recorded for each filament size, and the number of responses (0-3) was recorded. If the rat did not respond to the filament or responded only once, the next larger filament in the kit was applied and the process was repeated until the rat responded to at least two out of three applications. If the rat responded two or three times to the 4.31 hair, the smallest hair in the standard range (3.61) was applied, after which the process continued as above. Data was entered into the “PsychoFit” program (Harvey L O, University of Colorado at Boulder), which generated a 50% paw withdrawal threshold. This number was converted to force in grams and reported as the absolute threshold. Measurements were done at days 7, 10, 24 and 35 which correspond to day of first intra-articular injection of tested materials, 3 days after first injection, 3 and 14 days after second injection, respectively.

Gait Analysis:

Gait analysis was performed by applying ink to the ventral surface of the foot and documenting weight bearing during movement (footprints) across paper. Rear feet of rats were placed in ink, and then rats were placed on paper and allowed to walk the full length. This process was repeated as necessary to generate 4 clear, evenly inked footprint pairs representing the overall pattern of gait. Gait was scored visually from 0 to 6 where “0” refers to normal weight bearing and “6” refers to hopping, i.e., leg carrying (slight limp/pain=1, mild limp/pain=2, moderate limp/pain=3, marked limp/pain=4, severe limp/pain=5). Gait analysis footprints were analyzed digitally using ImageJ processing program to measure the area of the ink on a 300 dpi black and white scan. The image was smoothed, then the threshold was set at 0 (low) and 254 (high). The analyze particles function was used for the actual measurement, with size set to 0-Infinity and circularity set to 0-1. The values were reported in square inches, and the area of the right footprint was divided by the average of both footprints to determine the gait deficiency for each pair of prints.

Deficiency percentages approximate the clinical presentations described by the scores as follows: 0-5%=0; 6-15%=1; 16-30%=2; 31-50%=3; 51-75%=4; 76-99%=5; 100%=6.

DNA Nano-Plex Formation:

PolyJet™ transfection agent (PolyJet™, SignaGen) was added to commercial pmax-GFP plasmids at a 1:4 ratio (1 μg plasmid and 4 μl PolyJet™). Nano-complexes were formed in serum-free medium after 15 minutes of incubation at room temperature. In some cases, PolyJet™ was mixed with LABEL IT-Cy™3, at a ratio of 1:4 (0.5 μg non-labeled plasmid and 0.5 μg LABEL IT-Cy™3 and 4 μl PolyJet™). Nano-complexes were formed as above.

PEI (polyethylenimine) transfection agent was added to LABEL IT-Cy™3 plasmid and non-labeled plasmid at a 1:20 N/P ratio and at 0.5 μg from each plasmid per transfection. Nano-complexes were formed in serum-free medium after 15 minutes of incubation at room temperature.

Microscopic Imaging:

Images were taken using Nis-Elements F3.00 software (Nikon) and a Digital Sight digital camera (Nikon) from an Eclipse TS100 inverted fluorescence microscope (Nikon) supported with X-Cite® fluorescence illumination system (EXFO).

Statistical Analysis:

Statistical analysis was performed using Microsoft Excel statistical analysis software. Comparisons between two treatments were made using a student's T-test (two-tailed, equal variance). A p-value of <0.05 was considered to be statistically significant.

### Example 1

### Binding of F127-Fibrinogen Conjugate to Damaged Cartilage Surface

Circular cartilage explants were prepared from femoropatellar joints of freshly slaughtered bovine using a scalpel and 3 mm steel biopsy punch. Circular abrasions were then made on the surface of the explants using a 1.5 mm steel biopsy punch. The explants where then incubated for 3 days in 1 ml of chondrogenic medium (high glucose DMEM (Dulbecco's modified Eagle medium) +0.2% bovine serum albumin) containing 0.2 ml of FITC-labeled F127-fibrinogen prepared as described in the Materials and Methods section hereinabove. The explants were washed 3 times in PBS (twice in 1 ml and once in 25 ml, for 5 hours) and then fixed in 4% formaldehyde. Explants were then visualized using phase-contrast and fluorescent microscopy.

As shown in FIGS. 2A and 2B, fluorescent-labeled F127-fibrinogen associated specifically with damaged cartilage surfaces (abrasions) as opposed to intact cartilage surfaces.

These results indicate that the polymer-protein conjugates have a specific affinity to damaged cartilage surfaces.

### Example 2

### Effect of F127-Fibrinogen on Chondrocyte Pellet Model of Inflamed Cartilage

Ovine chondrocytes were cultured (as described hereinabove), and pellets were prepared using harvested monolayer chondrocytes (0.5×10⁶ cells per pellet). Cells were centrifuged at 1000 rpm (rotations per minute) for 5 minutes, counted, and re-suspended at a concentration of 10⁶ cells/ml in chondrogenic medium (high glucose DMEM, 10% fetal bovine serum, penicillin/streptomycin, 210 μM ascorbic acid (40 μg/ml), 10⁻⁷ M dexamethasone, 10 ng/ml TGF-β3) and divided among 15 ml conical tubes (0.5 ml in each tube). The tubes were centrifuged at 2000 rpm (500 g) for 10 minutes. The tubes lids were then left semi-open to allow gas exchange during a 3 weeks incubation (37° C., 5% CO₂), with medium replacements being performed every 3-4 days. At the end of 3 weeks, mature pellets were used for subsequent experiments.

For an in vitro inflammation model, the pellets were washed twice in PBS and 0.5 ng/ml of IL-1β (interleukin-1β in serum-free medium was added to the mature pellet in 3 doses. A first dose was added in serum-free medium for 4 days to create initial inflammation. The second and the third doses were added at 2 day intervals in the presence or absence of F127-fibrinogen (prepared as described hereinabove). To treat pellets with F127-fibrinogen, F127-fibrinogen (60 μl) was layered on top of each pellet followed by serum-free medium (120 μl) supplemented with IL1-β (at a final concentration of 0.5 ng/ml). Negative control samples received 180 μl of medium with 0.5 ng/ml IL1-β. The second and the third doses were added in the same manner after removing the previous medium and gel with a pipette.

sGAG (sulfated glycosaminoglycan) levels were quantified by dimethylmethylene blue (DMMB) assay and normalized to DNA content according to procedures described by Hoemann et al. [Anal Biochem 2002, 300:1-10]. The fixed histological cross-sections were stained using antibodies against collagen II or human fibrin.

As shown in FIG. 3, F127-fibrinogen formed a layer around the pellets that was tightly adhered to the pellets surface (as it was resistant to extensive washes).

As shown in FIG. 4, IL-1β induced a reduction in collagen type II (a component of cartilage ECM), which was reversed by F127-fibrinogen but not by Synvisc-One® viscosupplement.

In addition, as shown in FIG. 5, F127-fibrinogen completely reversed the IL-1β-mediated reduction in levels of sGAG.

These results indicate that that the polymer-protein conjugates provide protection against inflammation (by forming a protective layer) which is not provided by hyaluronic acid-based viscosupplements.

### Example 3

### Effect of F127-Fibrinogen on Dilution and Degradation of Hyaluronic Acid

Water uptake was compared among F127-fibrinogen, Synvisc-One® cross-linked hyaluronic acid viscosupplement, Arthrease® non-cross-linked hyaluronic acid viscosupplement, and 1:1 mixtures of F127-fibrinogen with Synvisc-One® or Arthrease® viscosupplement.

0.3 ml of tested material was placed in a 1.5 ml Eppendorf tube and the initial mass was recorded. The tubes were placed for 15 minutes in an incubator at 37° C. to enable gelation. After a gel was formed, 1 ml of PBS (pH 7.4, 37° C.) was added to each tube, and the tubes were sealed. Following incubation, the PBS was poured out and the final gel mass was recorded. The water uptake was calculated as a percentage using the following equation: 100×mass(final)/mass(initial). Shear storage modulus (G′) was measured as described in the Materials and Methods section.

In some samples, hyaluronidase was added in order to evaluate the effect of F127-fibrinogen in the presence of hyaluronidase, which fragments hyaluronic acid and is associated with synovial inflammation [Nagaya et al., Ann Rheum Dis 1999, 58:186-188].

As shown in FIGS. 6A and 6B, both cross-linked and non-cross-linked hyaluronic acid-based viscosupplements exhibited significant water uptake upon incubation in PBS for 48 hours at body temperature, whereas F127-fibrinogen exhibited no water uptake or negative water uptake (i.e., expulsion of water) under the same conditions (−13% water uptake in FIG. 6A, −1% in FIG. 6B). As further shown therein, mixtures of F127-fibrinogen with either type of hyaluronic acid-based viscosupplement resulted in significantly reduced water uptake (11% water uptake for mixture with cross-linked viscosupplement, 9% for mixture with non-cross-linked viscosupplement) in comparison with hyaluronic acid-based viscosupplement alone (50% water uptake for pure cross-linked viscosupplement, 25% for cross-linked viscosupplement).

As shown in FIG. 7, a mixture of F127-fibrinogen with Synvisc-One® exhibited an initial (t=0) G′_{Max} similar to that of pure Synvisc-One®, but after 48 hours, the G′_{Max} of the mixture decreased by only 25%, as compared with a 57% reduction for pure Synvisc-One®.

As further shown therein, in the presence of hyaluronidase, the G′_{Max} of pure Synvisc-One® decreased by 98%, whereas the G′_{Max} of the F127-fibrinogen/Synvisc-One® mixture decreased by 72%.

These results indicate that the polymer-protein conjugates reduce dilution of viscosupplements as well as the reduction in mechanical properties of the viscosupplements due to dilution or enzymatic degradation.

### Example 4

### Effect of F127-Fibrinogen on Coefficient of Friction

Lubrication by polymer-protein conjugates was assessed by comparing coefficients of friction (CoF; μ) for F127-fibrinogen and Synvisc-One® viscosupplement, using procedures described in the Materials and Methods section hereinabove.

As shown in FIG. 8, F127-fibrinogen exhibited a static CoF (μ=0.043) which was less than 20% of that exhibited by Synvisc-One® viscosupplement (μ=0.256).

The abovementioned static CoF for F127-fibrinogen was quite close to the value for normal synovial fluid (μ˜0.02), as reported by Ludwig et al. [Arthritis Rheum 2012, 64:3963-3971] and Ballard et al. [J Bone Joint Surg Am 2012, 94:e64]).

Similarly, as shown in FIG. 9, F127-fibrinogen exhibited a kinetic CoF which was considerably lower than that of Synvisc-One® viscosupplement under all measured sliding velocities.

These results indicate that the polymer-protein conjugates exhibit greater lubrication in comparison with conventional viscosupplements.

Without being bound by any particular theory, it is believed that protein (e.g., fibrinogen) moieties in the conjugate molecules facilitate the adhesion to cartilage surfaces, especially damaged cartilage surfaces (e.g., as exemplified hereinabove), and the synthetic polymer (e.g., F127 poloxamer) moieties provide enhanced lubrication, as depicted in FIG. 10, thereby providing a synergistic combination of adhesive and lubricating properties.

### Example 5

### Effect of F127-Fibrinogen on Cartilage Degeneration and Pain In Vivo

An in vivo rat model of (medial meniscal tear) osteoarthritis was used in order to assess the effects of F127-fibrinogen arthritic joints. In this model (35 day duration), damage to the meniscus induces progressive cartilage degeneration and osteophyte formation that mimic the changes that occur in spontaneous osteoarthritis.

Animals were anesthetized with isoflurane and the right knee area was prepared for surgery. A skin incision was made over the medial aspect of the knee and the medial collateral ligament was exposed by blunt dissection, and then transected. The medial meniscus was cut through the full thickness to simulate a complete tear. Skin and subcutis were closed with 4-0 Vicryl® suture. The model animals developed cartilage degeneration in the tibia. 7 days after surgery, the animals were dosed (by intra-articular injections) and evaluated as indicated in Table 1 below and in FIG. 11. The animals were sacrificed on day 35 and tissues were taken for histology. Treatment information was blinded until after the completion of histopathology.

TABLE 1
Treatments in different experimental groups

| Group No. | Treatment (two injections |
|---|---|
| n = 10 in | (20 μl each) into right |
| each group | knee joint with 7-day interval) |
| 1 | GelrinV (G′ = 10 Pa) |
| 2 | Synvisc-One ® viscosupplement |
| 3 | Phosphate buffer saline |
| 4 | GelrinV (G′ = 50 Pa) |

Following three days in 10% formic acid, the operated joints were cut into two approximately equal halves in the frontal plane and embedded in paraffin. Three sections were cut from each right knee at approximately 200 μm steps and stained with toluidine blue. A single section was cut from each left knee. Tissues were analyzed microscopically. The worst-case scenario for the two halves on each slide was determined and used for evaluation. The values for each parameter were then averaged across the three sections to determine overall values for each animal.

The width of degenerated cartilage was measured at location in which the damage was at its most severe form (“substantial”), i.e., maximal collagen and proteoglycan loss.

Significant cartilage degeneration was identified by chondrocyte and proteoglycan loss extending through greater than 50% of the cartilage thickness, and the precise width of degenerated cartilage was measured by ocular micrometer. In general, the collagen damage was mild (25% depth) or greater for this parameter but chondrocyte and proteoglycan loss extended to at least 50% or greater of the cartilage depth, indicating regions in which permanent structural changes have occurred.

As shown in FIGS. 12 and 13, the width of substantial cartilage degeneration in animals treated with F127-fibrinogen was lower than that of both control (PBS-treated) animals (by 13%) and Synvisc-One®-treated animals (by 11%).

In addition, as shown in FIG. 14, F127-fibrinogen formed a layer in vivo in association with cartilage surface.

The above results indicate that the polymer-protein conjugates can reduce cartilage degeneration, and suggests that such an effect may be mediated by forming an adherent layer on injured cartilage, which may lubricate the cartilage and/or act as a barrier to pro-inflammatory cytokines.

The effect of the treatments on pain in the rats was assessed by evaluation mechanical allodynia (pain due to a stimulus that does not normally provoke pain) and analysis of gaits of the animals (quantified as gait scores and gait deficiency percentages), as described in the Materials and Methods section (at the time points indicated in FIG. 11).

As shown in FIG. 15, both Synvisc-One® viscosupplement and F127-fibrinogen reduced sensitivity to secondary pain in operated joints, as evidenced by an increased threshold values over the course of day 7 to day 35.

As further shown in FIGS. 16A and 16B, F127-fibrinogen reduced gait score and gait deficiency, indicating increased weight bearing on an injured leg, in comparison to both control animals and Synvisc-One®-treated animals.

These results indicate that the polymer-protein conjugates reduce pain associated with arthritic joints, and are more effective in this respect than hyaluronic acid-based visco supplements.

### Example 6

### Properties of Mixtures of F127-Fibrinogen With Blood Fractions

Platelet rich plasma (PRP) and platelet poor plasma (PPP) were prepared according to procedures described by Nagata et al. [Eur J Dent 2010, 4:395-402]. Briefly, 3 ml of fresh blood sample from a healthy volunteer, with sodium citrate, was centrifuged at 160 g for 6 minutes at room temperature. 0.6 ml of PPP (top layer) was then pipetted. Next, a mark was made 2 mm below the line that separates the middle component from lower component of the tube. All content above this point (approximately 0.7 ml) was pipetted and comprised the PRP component.

For rheological measurements, 150 μl of PRP or PPP were mixed with 150 μl of GelrinV (10 mg/ml fibrinogen) at a temperature below 20° C., to obtain a homogeneous solution that was kept on ice. No precipitation or coagulation occurred upon mixing. As a control, GelrinV was mixed with PBS at a ratio of 1:1. 200 μl samples of the mixtures were used for temperature-dependent rheological measurements.

As shown in FIG. 17, F127-fibrinogen exhibited reverse thermal gelation properties (markedly increased G′ values at higher temperatures) when mixed at a 1:1 ratio with blood fractions (non-activated platelet-rich plasma and platelet-poor plasma). As further shown therein, reverse thermal gelation of the mixtures with blood fractions were characterized by higher G′ values and by lower gelation temperature than reverse thermal gelation of the mixture with PBS, indicating that interactions between the F127-fibrinogen and blood fractions enhance gelation.

These results indicate that compositions comprising polymer-protein conjugates can serve as a carrier for blood fractions (e.g., autologous blood fractions), for example, for allowing continuous release of growth factors from encapsulated platelets (e.g., in order to promote cartilage repair).

### Example 7

### Gene Delivery Using F127-Fibrinogen Composition

In order to demonstrate retention of DNA nano-complexes over time within a polymer-protein conjugate composition, 300 μl of GelrinV (8 mg/ml fibrinogen) was mixed at 4° C. with DNA nano-complexes prepared from PolyJet™ or PEI transfection reagents with non-labeled and Cy3-labeled plasmid as described above, or with naked plasmid DNA (1 μg plasmid in 100 ∥l). The DNA-containing GelrinV was then mixed with a C2C12 cell pellet (containing 10⁶ cells) and incubated at 37° C. in 48-well tissue culture plate for 40 minutes followed by addition of growth medium. At each time point indicated herein, images were taken using a fluorescence microscope.

As shown in FIG. 18, naked Cy3-plasmid DNA diffused out of the gel, whereas nano-complexes made with PolyJet™ and PEI transfection reagents remained in the gel 48 hours post encapsulation.

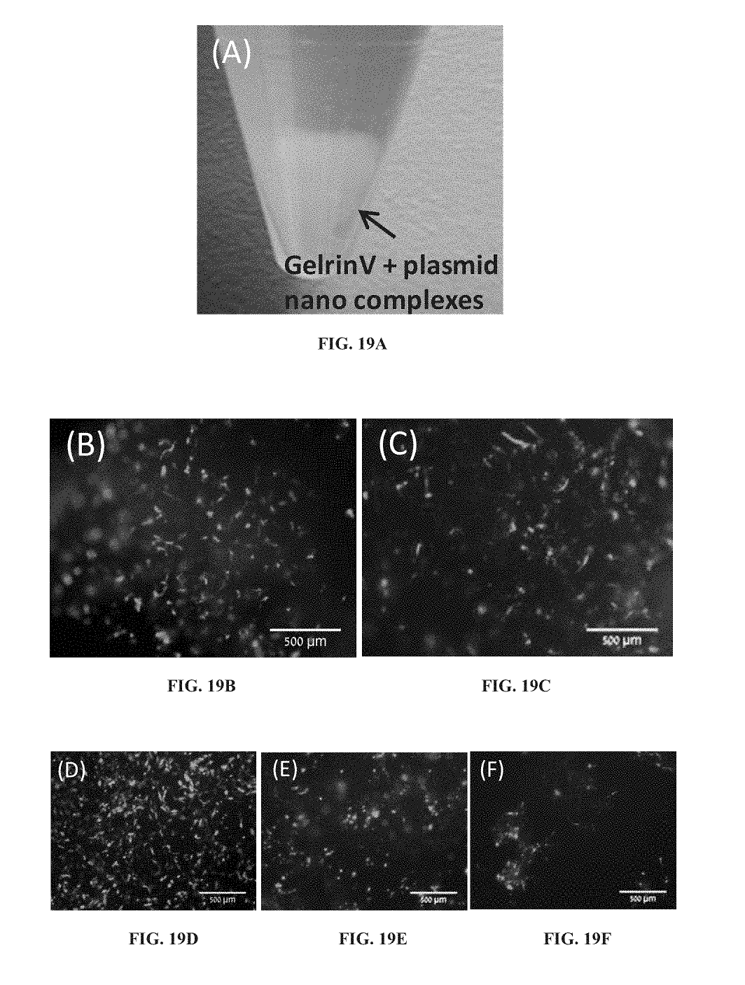

DNA nano-complexes (nano-plexes) were prepared as described in the Materials and Methods section hereinabove, using plasmids for GFP (green fluorescent protein), and mixed with GelrinV (shown in FIG. 19A), and gene delivery using the GelrinV-plasmid mixture was assessed under a variety of conditions.

To perform 3D (encapsulated cell) gene delivery, in some cases C2C12 myoblasts were pre-incubated with DNA nano-plexes for 20 minutes, mixed with GelrinV at room temperature (5×10⁶ cells per ml gel) and then a gel was formed upon incubation at 37° C. for 40 minutes (FIG. 19B). In other cases, the cells and nano-plexes were mixed without pre-incubation with GelrinV and gel was formed as described above (FIG. 19C).

To perform 2D (adjacent cell) gene delivery, in some cases, GelrinV containing nano-plexes was layered on top of cells that were pre-adhered to tissue culture plastic (FIG. 19D). In other cases, GelrinV gel was mixed with nano-plexes, pre-polymerized in a 15 ml tube or in a non-adherent tissue culture plates at 37° C. for 40 minutes and cells seeded on top (FIG. 19E).

Delivery of GFP plasmid was assessed by microscopic observations using standard fluorescent microscope with fluorescein isothiocyanate filter.

As shown in FIGS. 19B-19F, successful 2D (FIGS. 19B and 19C) and 3D (FIGS. 19D-19F) gene delivery was achieved using a F127-fibrinogen composition for DNA nano-plex delivery, as evidenced by a relatively high number of GFP-expressing cells.

GelrinV samples (100 μl) containing GFP plasmid nano-plexes were subjected (or not subjected) to two washes (5 ml each) with PBS before cell seeding in 2D configuration (as described above).

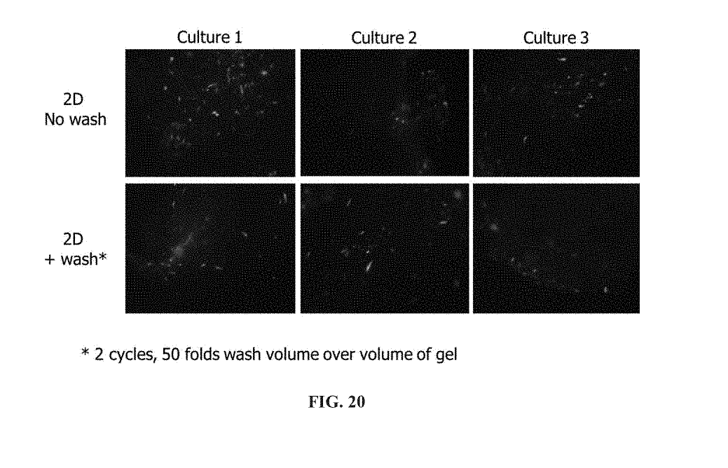

As shown in FIG. 20, the transfection efficiency was not reduced by washes. This result indicates that the gene delivery was not due to burst released nano-complexes but rather due to encapsulated nano-complexes.

These results indicate that polymer-protein conjugates are suitable for facilitating gene delivery. Importantly, the ability of GelrinV to deliver plasmid DNA to cells in 2D facilitates its use in vivo in a cell-free configuration.

### Example 8

### Effect of F127-Fibrinogen Composition on Bone Cyst

The GelrinV composition (prepared as described hereinabove) is injected into a bone cyst (in a human subject), optionally a subchondral bone cyst. Computed tomography (CT) imaging of the bone cyst region is optionally performed prior to injection and several months after injection, in order to assess cyst filling. In addition, pain assessment is optionally performed prior to injection and several months after injection by an accepted technique, e.g., using an 11-point numeric visual analog scale (VAS). Enhancement of bone cyst filling and/or reduction in pain (e.g., relative to control group) are quantified.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a condition associated with degeneration of articular cartilage and/or with subchondral bone loss in a subject in need thereof, the method comprising administering to the subject a composition comprising a conjugate which comprises a polypeptide having attached thereto at least two polymeric moieties, at least one of said polymeric moieties exhibiting a reverse thermal gelation, thereby treating the condition.

2. The method of claim 1, comprising intra-articular administration of the composition.

3. (canceled)

4. The method of claim 1, wherein said degeneration is associated with friction at a surface of said articular cartilage.

5. (canceled)

6. The method of claim 1, wherein said degeneration of articular cartilage and/or said subchondral bone loss is associated with an inflammation.

7. The method of claim 1, wherein said condition is associated with a subchondral bone cyst.

8. (canceled)

9. The method of claim 1, wherein the composition is characterized by water uptake of less than 20 weight percents upon incubation with an aqueous liquid for 48 hours at a temperature of 37° C.

10. The method of claim 1, wherein the composition comprises an aqueous solution of said conjugate.

11. (canceled)

12. The method of claim 1, wherein the composition is capable of undergoing a reverse thermal gelation.

13. The method of claim 1, wherein the composition further comprises at least one additional therapeutically active agent.

14. (canceled)

15. The method of claim 1, wherein said condition is arthritis.

16. (canceled)

17. The method of claim 1, wherein at least a portion of said articular cartilage and/or said subchondral bone is in a synovial joint.

18. The method of claim 1, wherein said polypeptide is at least 20 amino acids in length.

19. The method of claim 1, wherein said polypeptide is capable of adhering to cartilage.

20. (canceled)

21. The method of claim 1, wherein said polypeptide comprises a protein or a fragment thereof.

22. (canceled)

23. The method of claim 1, wherein each of said polymeric moieties exhibits a reverse thermal gelation.

24-25. (canceled)

26. The method of claim 1, wherein at least one of said polymeric moieties further comprises at least one cross-linking moiety capable of covalently cross-linking said conjugate with a protein in vivo.

27. A pharmaceutical composition comprising:
a conjugate which comprises a polypeptide having attached thereto at least two polymeric moieties, at least one of said polymeric moieties exhibiting a reverse thermal gelation; and
at least one additional therapeutically active agent selected from the group consisting of a hyaluronic acid, a blood fraction, and a cell,
the composition being an aqueous composition which forms a hydrogel at a temperature in a range of from 32° C. to 37° C.

28. (canceled)

29. The composition of claim 27, being characterized by water uptake of less than 20 weight percents upon incubation with an aqueous liquid for 48 hours at a temperature of 37° C.

30. (canceled)

31. The composition of claim 27, being capable of undergoing a reverse thermal gelation.

32. The composition of claim 27, wherein at least 20 weight percents of the composition is said blood fraction.

33. (canceled)

34. The composition of claim 27, wherein said polypeptide is at least 20 amino acids in length.

35. The composition of claim 27, wherein said polypeptide comprises a protein or a fragment thereof.

36. (canceled)

37. The composition of claim 27, wherein each of said polymeric moieties exhibits a reverse thermal gelation.

38-39. (canceled)

40. The composition of claim 27, wherein at least one of said polymeric moieties further comprises at least one cross-linking moiety capable of covalently cross-linking said conjugate with a protein in vivo.

41. The composition of claim 27, being an injectable composition.

42. (canceled)

43. A method of treating a condition in a subject in need thereof, the method comprising administering to the subject the composition of claim 27, wherein said condition is treatable by said therapeutically active agent, thereby treating the condition.

44. The method of claim 43, wherein said condition is treatable by local administration of said therapeutically active agent, and the method comprises local administration of the composition.

45. The method of claim 43, wherein said at least one therapeutically active agent comprises said blood fraction, and said condition is selected from the group consisting of arthritis, nerve injury, tendinitis, muscle injury, bone injury, and surgical injury.

46. (canceled)

47. The method of claim 43, wherein said at least one therapeutically active agent comprises said hyaluronic acid, and said condition is arthritis.

48. A method of effecting gene delivery, the method comprising contacting at least one cell with athe composition comprising:
a conjugate which comprises a polypeptide having attached thereto at least two polymeric moieties, at least one of said polymeric moieties comprising a poloxamer (poly(ethylene oxide-propylene oxide) copolymer) and exhibiting a reverse thermal gelation; and
a nucleic acid comprising said gene,
the composition being an aqueous composition which forms a hydrogel at a temperature in a range of from 32° C. to 37° C.,
thereby effecting delivery of said gene to said at least one cell.

49-50. (canceled).
